(19) United States
(12) Patent Application Publication
(10) Pub. No.: US 2012/0030784 A1
(43) Pub. Date: Feb. 2, 2012

(54) BETA-GLUCAN-DEFICIENT GENE IN BARLEY, SYNTHETIC GENE, AND USE OF SAME

(75) Inventors: Takuji Tonooka, Ibaraki (JP); Toji Yoshioka, Ibaraki (JP); Emiko Aoki, Ibaraki (JP); Shin Taketa, Okayama (JP)

(73) Assignee: Incorporated Administration Agency National Agriculture and Food Research Organization, Tsukuba-shi (JP)

(21) Appl. No.: 13/203,675

(22) PCT Filed: Feb. 25, 2010
(86) PCT No.: PCT/JP2010/052939

§ 371 Date: Oct. 11, 2011

(30) Foreign Application Priority Data
Feb. 27, 2009 (JP) 2009-045393

(51) Int. Cl. A01H 1/02; A01H 1/04; A01H 5/00; A23K 1/00; C12N 1/21; C12N 1/19; A01H 1/06; C12N 15/29; C12P 7/02

(52) U.S. Cl. 800/260; 536/23.6; 800/284; 800/320; 435/155; 435/252.33; 435/254.2; 435/254.21; 435/254.23; 800/276; 426/18; 426/636

(57) ABSTRACT

A Genomic DNA and a cDNA of a β-glucan-deficient gene in a barley and of a β-glucan synthesis gene in a barley, a barley having the genomic DNA of a β-glucan-deficient gene and a method for breeding the same, a method for producing an alcohol or a fermented food using barley kernels decreasing or deficient in β-glucan, and an animal feed composition are provided. A Genomic DNA of a β-glucan-deficient gene consisting of a base sequence having 90% or more homology to the base sequence of SEQ ID NO: 1 and producing a protein lacking a β-glucan synthesis activity when transcribed and translated, and a breeding method comprising a step of selecting a barley by determining the barley as having a β-glucan-deficient gene when the base corresponding to position 4,275, or corresponding to position 2,385, of the gene consisting of the base sequence of SEQ ID NO: 4 located near the centromere of chromosome 7H of the barley is mutated from G to A.

### TECHNICAL FIELD

The present invention relates to a gene deficient in (1-3, 1-4)-β-D-glucan (hereinafter referred to as “β-glucan”) (hereinafter referred to as a “β-glucan-deficient gene”) and a β-glucan synthesis gene in barley kernels and the use thereof. Specifically, the invention relates to: genomic DNA and cDNA of a β-glucan-deficient gene in a barley; genomic DNA and cDNA of a β-glucan synthesis gene in a barley; a barley having the genomic DNA of a β-glucan-deficient gene and a method for breeding the same; a method for decreasing or abolishing β-glucan in barley kernels by suppressing the expression of the β-glucan synthesis gene and a barley having a decrease or deficiency in β-glucan in kernels thereof, obtainable by the method; a method for producing an alcohol or a fermented food, comprising a step of fermenting kernels of the barley decreasing or deficient in β-glucan; and an animal feed composition containing the barley kernels. Here, the β-glucan synthesis gene means a gene encoding a protein having the function of participating in the pathway for the synthesis of β-glucan and promoting the synthesis of β-glucan.

### BACKGROUND ART

Unlike cereals such as wheat and rice, barley kernels are rich in β-glucan as one of polysaccharides. β-Glucan is the major component constituting the endosperm cell wall thereof. It has been shown that a high content of β-glucan decreases the saccharification of starch and the efficiency of fermentation in the brewing of beer and shochu (a Japanese distilled spirit) and has negative effects such as poor digestion, poor absorption and a low feeding effect. Thus, in the barley used for these applications, there is a need for the lower content of β-glucan; a gene deficient in β-glucan is useful.

β-Glucan has also been shown to have physiological effects such as the suppression of the increased blood glucose level and the reduction of blood cholesterol and be effective in preventing and improving lifestyle-related diseases. It has been reported to be also effective in improving the immune function in recent years (Non Patent Literature 1). Thus, when barley is used as food, that having a higher content of β-glucan has a higher use value.

### CITATION LIST

### Non Patent Literature

Non Patent Literature 1

Brennan, C. S. and L. J. Cleary (2005) J. Cereal Sci. 42: 1-13.

### SUMMARY OF INVENTION

### Technical Problem

In order to genetically control the β-glucan content for breeding, it is essential to elucidate the gene involved in the synthesis and control of β-glucan. The gene involved in β-glucan synthesis remains to be elucidated despite that β-glucan is an important component constituting the cell wall of a plant while being a highly functional polysaccharide attracting world-wide attention. The elucidation of the gene becomes severely competitive on a worldwide scale.

An object of the present invention is to provide genomic DNA and cDNA of a β-glucan-deficient gene in a barley. Another object of the present invention is to provide genomic DNA and cDNA of a β-glucan synthesis gene in a barley. A further object of the present invention is to provide a barley having the genomic DNA of a β-glucan-deficient gene and a method for breeding the same, a method for producing an alcohol, comprising a step of fermenting kernels of the barley, and a an animal feed composition containing the barley kernels.

### Solution to Problem

In one aspect, the present invention provides a genomic DNA of a β-glucan-deficient gene which is a DNA consisting of a base sequence having 90% or more homology to the base sequence of SEQ ID NO: 1 and producing a protein lacking a β-glucan synthesis activity when transcribed and translated.

In another aspect, the present invention provides a genomic DNA of a β-glucan-deficient gene which is a DNA consisting of a base sequence having 90% or more homology to the base sequence of SEQ ID NO: 18 and producing a protein lacking a β-glucan synthesis activity when transcribed and translated.

The genomic DNA of a β-glucan-deficient gene can be used for producing a barley in whose kernels the aleurone layer and the endosperm cell wall are completely deficient in β-glucan and the endosperm cell wall is noticeably thin. The use of barley kernels deficient in β-glucan as a raw material can enhance the efficiency of saccharification of starch and fermentation in the production of beer and shochu. An animal feed composition for pigs, fowls, or the like, comprising barley kernels deficient in β-glucan is also excellent in digestion absorption and has a high feeding effect. In addition, the barley deficient in β-glucan contains slightly more arabinoxylan important as a functional polysaccharide; thus, it can be used for the development of a functional food specialized in arabinoxylan. Although it is generally difficult to separate β-glucan and arabinoxylan, pure arabinoxylan can be obtained by extracting arabinoxylan from the barley deficient in β-glucan.

The protein lacking a β-glucan synthesis activity produced when the genomic DNA of a β-glucan-deficient gene of the present invention is transcribed and translated is more preferably a protein lacking a β-glucan synthesis activity consisting of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is an amino acid other than glycine. The amino acid corresponding to position 660 is more preferably aspartic acid. In addition, the genomic DNA of a β-glucan-deficient gene of the present invention particularly preferably consists of the base sequence of SEQ ID NO: 1.

Alternatively, the protein lacking a β-glucan synthesis activity produced when the genomic DNA of a β-glucan-deficient gene of the present invention is transcribed and translated is more preferably a protein lacking a β-glucan synthesis activity consisting of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is an amino acid other than cysteine. The amino acid corresponding to position 253 is more preferably tyrosine. In addition, the genomic DNA of a β-glucan-deficient gene of the present invention particularly preferably consists of the base sequence of SEQ ID NO: 18.

In another aspect, the present invention provides a cDNA of a β-glucan-deficient gene encoding a protein lacking a β-glucan synthesis activity consisting of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is an amino acid other than glycine.

In another aspect, the present invention provides a cDNA of a β-glucan-deficient gene encoding a protein lacking a β-glucan synthesis activity consisting of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is an amino acid other than cysteine.

The cDNA of a β-glucan-deficient gene is involved in the synthesis of β-glucan; thus, it can be used for the elucidation of a plant gene involved in the synthesis of β-glucan in the field of life science. If the gene involved in the synthesis of β-glucan is elucidated, the results can be used to genetically control the content of β-glucan in breeding.

In the amino acid sequence encoded by the cDNA of a β-glucan-deficient gene according to the present invention, the amino acid corresponding to position 660 of the amino acid sequence of SEQ ID NO: 2 is more preferably aspartic acid. The cDNA of a β-glucan-deficient gene according to the present invention also particularly preferably consists of the base sequence of SEQ ID NO: 3.

Alternatively, in the amino acid sequence encoded by the cDNA of a β-glucan-deficient gene according to the present invention, the amino acid corresponding to position 253 of the amino acid sequence of SEQ ID NO: 20 is more preferably tyrosine. The cDNA of a β-glucan-deficient gene according to the present invention also particularly preferably consists of the base sequence of SEQ ID NO: 19.

In another aspect, the present invention provides a genomic DNA of a β-glucan synthesis gene which is DNA consisting of a base sequence having 90% or more homology to the base sequence of SEQ ID NO: 4 and producing a protein having a β-glucan synthesis activity when transcribed and translated. The genomic DNA of a β-glucan synthesis gene particularly preferably consists of the base sequence of SEQ ID NO: 4.

In another aspect, the present invention provides a genomic DNA of a β-glucan synthesis gene which is DNA consisting of a base sequence having 90% or more homology to the base sequence of SEQ ID NO: 15 and producing a protein having a β-glucan synthesis activity when transcribed and translated. The genomic DNA of a β-glucan synthesis gene particularly preferably consists of the base sequence of SEQ ID NO: 15.

Although the gene involved in the synthesis of β-glucan has not been elucidated, the present inventors have determined that the above DNAs are involved in β-glucan synthesis. It is possible to develop a plant variety showing a high production level of β-glucan by a method such as increasing the number of copies of the genomic DNA of a β-glucan synthesis gene in the plant. β-Glucan has a higher use value because it has physiological effects such as the suppression of the increased blood glucose level and the reduction of blood cholesterol and effects such as the promotion of immune function.

In another aspect, the present invention provides a cDNA of a β-glucan synthesis gene which is DNA consisting of a base sequence having 90% or more homology to the base sequence of SEQ ID NO: 6 and encoding a protein having a β-glucan synthesis activity. The cDNA of a β-glucan synthesis gene particularly preferably consists of the base sequence of SEQ ID NO: 6.

Although the gene involved in the synthesis of β-glucan has not been elucidated, it has been determined according to the present invention that the above cDNA is involved in β-glucan synthesis. The cDNA can be introduced into a bacterium, a yeast, a plant, or the like by linking downstream of a suitable promoter to produce β-glucan on a large scale. β-Glucan has a high use value because it has physiological effects such as the suppression of the increased blood glucose level and the reduction of blood cholesterol and effects such as the promotion of immune function.

In another aspect, the present invention provides a method for breeding a barley having a genomic DNA of a β-glucan-deficient gene, comprising a selection step of selecting the barley by determining the barley as having a genomic DNA of a β-glucan-deficient gene when the base corresponding to position 4,275 of the genomic DNA consisting of the base sequence of SEQ ID NO: 4 located near the centromere of chromosome 7H of the barley is mutated from G (guanine) to A (adenine)

In another aspect, the present invention provides a method for breeding a barley having a genomic DNA of a β-glucan-deficient gene, comprising a selection step of selecting the barley by determining the barley as having a genomic DNA of a β-glucan-deficient gene when the base corresponding to position 2,385 of the genomic DNA consisting of the base sequence of SEQ ID NO: 4 located near the centromere of chromosome 7H of the barley is mutated from G to A.

These breeding methods can be used to breed a barley having a genomic DNA of a β-glucan-deficient gene. Conventional methods for determining the presence of β-glucan have been methods which use a kernel as a specimen to detect a fluorochrome specifically adsorbing to, or a product resulting from an enzymatic treatment of β-glucan. Thus, in order to perform the above determination, it is required to grow a plant until the stage in which seeds are formed. In contrast, the use of the present invention enables the determination to be carried out even in an infant plant before the formation of seeds and also enables the determination of a homozygous or heterozygous genotype. Therefore, the period necessary for breeding can be remarkably shortened.

In one aspect of the above breeding method, the present invention provides a breeding method comprising: an amplification step of amplifying a DNA fragment containing a base corresponding to position 4,275 of the base sequence of SEQ ID NO: 4 using a genomic DNA extracted from a barley as a template; a detection step of cleaving the DNA fragment amplified in the amplification step with a restriction enzyme selected from the group consisting of TaqI, BanI, and NlaIV and detecting the cleaved DNA fragment; and a selection step of selecting the barley by determining the barley as having a genomic DNA of a β-glucan-deficient gene when the DNA fragment is cleaved with the restriction enzyme TaqI or not cleaved with the restriction enzyme BanI or NlaIV in the detection step.

The breeding method enables rapid determination when the base is mutated which corresponds to position 4,275 of the genomic DNA consisting of the base sequence of SEQ ID NO: 4. The use of the restriction enzyme BanI or NlaIV enables determination when the base corresponding to the position 4,275 is mutated from G to A, C (cytosine), or T (thymine). The use of the restriction enzyme TaqI enables determination when the base corresponding to the position 4,275 is mutated from G to A.

In one aspect of the above breeding method, the present invention provides a breeding method comprising: an amplification step of amplifying a DNA fragment containing a base corresponding to position 2,385 of the base sequence of SEQ ID NO: 4 using a genomic DNA extracted from a barley as a template; a detection step of cleaving the DNA fragment amplified in the amplification step with a restriction enzyme, Fnu4HI, and detecting the cleaved DNA fragment; and a selection step of selecting the barley by determining the barley as having a genomic DNA of a β-glucan-deficient gene when the DNA fragment is not cleaved between the bases corresponding to positions 2,386 and 2,387 of SEQ ID NO: 4 in the detection step.

The breeding method enables rapid determination when the base is mutated which corresponds to position 2,385 of the genomic DNA consisting of the base sequence of SEQ ID NO: 4. Thus, when the base is G, the amplified DNA fragment is cleaved between the bases corresponding to positions 2,386 and 2,387 of SEQ ID NO: 4 by Fnu4HI, indicating that the barley is wild type. When the base corresponding to position 2,385 of the genomic DNA consisting of the base sequence of SEQ ID NO: 4 is mutated from G to A, C, or T, the above amplified DNA fragment is not cleaved between the bases corresponding to positions 2,386 and 2,387 of SEQ ID NO: 4 by Fnu4HI, indicating that the barley has a genomic DNA of a β-glucan-deficient gene.

In another aspect, the present invention provides a barley bred by the above method, the barley having any of the DNAs:

(a) a genomic DNA of a β-glucan-deficient gene consisting of a base sequence having 90% or more homology to the base sequence of SEQ ID NO: 1 and producing a protein lacking a β-glucan synthesis activity when transcribed and translated;

(b) the genomic DNA of a β-glucan-deficient gene according to (a) above, wherein the protein produced in the transcription and translation consists of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is an amino acid other than glycine;

(c) the genomic DNA of a β-glucan-deficient gene according to (b) above, wherein the amino acid other than glycine is aspartic acid;

(d) a genomic DNA of a β-glucan-deficient gene, consisting of the base sequence of SEQ ID NO: 1;

(e) a genomic DNA of a β-glucan-deficient gene consisting of a base sequence having 90% or more homology to the base sequence of SEQ ID NO: 18 and producing a protein lacking a β-glucan synthesis activity when transcribed and translated;

(f) the genomic DNA of a β-glucan-deficient gene according to (e) above, wherein the protein produced in the transcription and translation consists of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is an amino acid other than cysteine;

(g) the genomic DNA of a β-glucan-deficient gene according to (f) above, wherein the amino acid other than cysteine is tyrosine; and

(h) a genomic DNA of a β-glucan-deficient gene, consisting of the base sequence of SEQ ID NO: 18. The barley of the present invention more preferably has any genomic DNA of a β-glucan-deficient gene of (a) to (h) above in homozygous form.

A barley having any genomic DNA of a β-glucan-deficient gene of (a) to (h) above in heterozygous form has decreased β-glucan in a kernel as compared to a wild-type barley; a barley having any genomic DNA of a β-glucan-deficient gene of (a) to (h) above in homozygous form is completely deficient in kernel β-glucan.

In another aspect, the present invention provides a method for decreasing or abolishing β-glucan in barley kernels, comprising a step of suppressing the expression of a genomic DNA of a β-glucan synthesis gene consisting of a base sequence having 90% or more homology to the base sequence of SEQ ID NO: 4 and producing a protein having a β-glucan synthesis activity when transcribed and translated or a genomic DNA of a β-glucan synthesis gene consisting of the base sequence of SEQ ID NO: 4 and a barley decreasing or deficient in β-glucan obtainable by the method. This barley decreases or is completely deficient in kernel β-glucan.

In another aspect, the present invention provides a method for decreasing or abolishing β-glucan in barley kernels, comprising a step of suppressing the expression of a genomic DNA of a β-glucan synthesis gene consisting of a base sequence having 90% or more homology to the base sequence of SEQ ID NO: 15 and producing a protein having a β-glucan synthesis activity when transcribed and translated or a genomic DNA of a β-glucan synthesis gene consisting of the base sequence of SEQ ID NO: 15 and a barley decreasing or deficient in β-glucan obtainable by the method. This barley decreases or is completely deficient in kernel β-glucan.

In another aspect, the present invention provides a method for producing an alcohol, comprising a step of fermenting kernels derived from a barley bred by the above breeding method, which is a barley having any genomic DNA of a β-glucan-deficient gene of (a) to (h) above or a barley decreasing or deficient in kernel β-glucan obtainable by suppressing the expression of the above genomic DNA of a β-glucan synthesis gene. A barley having genomic DNA of a β-glucan-deficient gene in heterozygous form has decreased β-glucan in a kernel as compared to a wild-type barley; a barley having that in homozygous form is completely deficient in kernel β-glucan. Barley kernels decreasing or deficient in β-glucan can be used as a raw material to provide effects such as the shortening of the saccharification time of starch, the shortening of filtration time of wort, the increase of the alcohol formation and the decrease of fermentation residues in the production of beer or shochu.

In another aspect, the present invention provides a method for producing a fermented food, comprising a step of fermenting kernels derived from a barley bred by the above breeding method, which is a barley having any genomic DNA of a β-glucan-deficient gene of (a) to (h) above or a barley decreasing or deficient in kernel β-glucan obtainable by suppressing the expression of the above genomic DNA of a β-glucan synthesis gene. The fermented food is a food comprising one obtained by fermenting barley kernels and, for example, a soybean paste or a soy sauce. Barley kernels decreasing or deficient in β-glucan can be used as a raw material to provide effects such as the shortening of the time necessary for fermentation because the efficiency of fermentation can be increased.

In another aspect, the present invention provides an animal feed composition comprising kernels derived from a barley bred by the above breeding method, which is a barley having any genomic DNA of a β-glucan-deficient gene of (a) to (h) above or a barley decreasing or deficient in kernel β-glucan obtainable by suppressing the expression of the above genomic DNA of a β-glucan synthesis gene. The animal feed composition comprising barley kernels decreasing or completely deficient in β-glucan is excellent for digestion and absorption and has a high feeding effect. The improved digestibility results in the obtaining of effects such as the decrease of the amount of feces. The kernel can be more easily ground in producing the feed composition because the endosperm cell wall is thin and the kernel is soft.

In another aspect, the present invention provides a transformant holding, so as to be expressible, a vector containing any of the DNAs:

(a) a genomic DNA of a β-glucan synthesis gene consisting of a base sequence having 90% or more homology to the base sequence of SEQ ID NO: 4 and producing a protein having a β-glucan synthesis activity when transcribed and translated;

(b) a genomic DNA of a β-glucan synthesis gene, consisting of the base sequence of SEQ ID NO: 4;

(c) a cDNA of a β-glucan synthesis gene consisting of a base sequence having 90% or more homology to the base sequence of SEQ ID NO: 6 and encoding a protein having a β-glucan synthesis activity;

(d) a cDNA of a β-glucan synthesis gene consisting of the base sequence of SEQ ID NO: 6;

(e) a genomic DNA of a β-glucan synthesis gene consisting of a base sequence having 90% or more homology to the base sequence of SEQ ID NO: 15 and producing a protein having a β-glucan synthesis activity when transcribed and translated; and

(f) a genomic DNA of a β-glucan synthesis gene consisting of the base sequence of SEQ ID NO: 15. The transformant may be a barley or a plant containing no β-glucan or containing β-glucan only in an extremely small quantity. Alternatively, the transformant may be a microorganism exemplified by a prokaryote such as Escherichia coli or a eukaryote such as yeast.

Such a transformant plant has high added value since it contains β-glucan in more abundance. Alternatively, such a transformant microorganism can also be used to industrially produce β-glucan in volume.

The transformant more preferably overexpresses the genomic DNA of the β-glucan synthesis gene or the cDNA of the β-glucan synthesis gene. The overexpression means expressing high amounts of the β-glucan synthesis gene at the mRNA level or the protein level compared to the host before transformation.

### Advantageous Effects of Invention

According to the present invention, a genomic DNA and a cDNA of a β-glucan-deficient gene in a barley are provided. A genomic DNA and a cDNA of a β-glucan synthesis gene in a barley are also provided. Further provided are: a barley having the genomic DNA of a β-glucan-deficient gene and a method for breeding the same; a method for decreasing or abolishing β-glucan in barley kernels by suppressing the expression of a β-glucan synthesis gene, and a barley decreasing or deficient in β-glucan in kernels thereof, obtainable by the method; a method for producing an alcohol or a fermented food, comprising a step of fermenting these barley kernels decreasing or deficient in β-glucan; and an animal feed composition comprising the barley kernels.

The modification of a gene involved in the synthesis of β-glucan is useful in the elucidation of a gene involved in the synthesis of β-glucan in a plant in the field of life science and the breeding of a barley variety containing no β-glucan in the field of agriculture. The elucidation of a β-glucan-deficient gene and a β-glucan deficiency mechanism contribute to the identification of a gene involved in the synthesis of β-glucan. A barley deficient in β-glucan is an important resource for the development of a functional food specialized in arabinoxylan because of containing a slightly large amount of arabinoxylan important as a functional polysaccharide together with β-glucan. Conventional methods for determining the presence of β-glucan have been methods which use a kernel as a specimen to detect a fluorochrome specifically adsorbing to, or a product resulting from an enzymatic treatment of β-glucan. Thus, in order to perform the above determination, it is required to grow a plant until the stage in which seeds are formed. In contrast, the use of the present invention enables the determination to be carried out even in an infant plant before the formation of seeds and also enables the determination of a homozygous or heterozygous genotype. The use of a β-glucan-deficient gene according to the present invention enables the production of a barley in which the aleurone layer and the endosperm cell wall are completely deficient in β-glucan and the endosperm cell wall is noticeably thin.

### BRIEF DESCRIPTION OF DRAWINGS

FIG. 1 is a diagram showing a position on which a β-glucan-deficient gene is located on a chromosome.

FIG. 2 is a diagram showing a base sequence of HvCslF6 gene and a deduced amino acid sequence therefrom in a β-glucan-deficient barley variety and a wild-type variety thereof.

FIG. 3 is a set of photographs showing an example of the determination of a HvCslF6 genotype using a CAPS marker.

FIG. 4 is a set of photographs showing observations of kernel fragments of a Nishinohoshi (bgl) near-isogenic line and Nishinohoshi under a light microscope.

FIG. 5 is a set of photographs showing observations of kernel fragments of a Nishinohoshi (bgl) near-isogenic line and Nishinohoshi under a light microscope and a scanning electron microscope.

FIG. 6 is a diagram showing promoter sequences of HvCslF6 genes in TR251 and CDC-Bold.

FIG. 7 is a diagram showing promoter sequences of HvCslF6 genes in TR251 and CDC-Bold.

FIG. 8 is a diagram showing base sequences of HvCslF6 genes in Sachiho Golden (TN5) and KM27. TN5-DNA indicates a portion of a base sequence of genomic DNA of a HvCslF6 gene (SEQ ID NO: 15) in Sachiho Golden. TN5-cDNA indicates a portion of a base sequence of cDNA of a HvCslF6 gene (SEQ ID NO: 16) in Sachiho Golden. TN5-a.a. indicates a portion of an amino acid sequence of HvCslF6 (SEQ ID NO: 17) in Sachiho Golden. KM27-DNA indicates a portion of a base sequence of genomic DNA of a HvCslF6 gene (bgl gene) (SEQ ID NO: 18) in KM27. KM27-cDNA indicates a portion of a base sequence of cDNA of bgl gene (SEQ ID NO: 19) in KM27. KM27-a.a. indicates a portion of an amino acid sequence (SEQ ID NO: 20) encoded by bgl gene in KM27.

FIG. 9 is a diagram showing a predicted three-dimensional structure of HvCslF6. (i) indicates a site at which cysteine is mutated to tyrosine in KM27. (ii) indicates a site at which glycine is mutated to aspartic acid in OUM125.

FIG. 10 is a photograph showing an example of the determination of a HvCslF6 genotype using a CAPS marker.

### DESCRIPTION OF EMBODIMENTS

As used herein, a “base sequence having 90% or more homology” means a base sequence whose bases are the same as 90% or more, preferably 95% or more, more preferably 98% or more, still more preferably 99% or more of the bases of another base sequence when these two base sequences are compared by alignment such that as many of their bases as possible coincide with each other. Here, in aligning the base sequences, gaps may be contained so as to provide the maximum homology.

As used herein, an “amino acid sequence in which one or several amino acids are deleted, substituted, or added” means an amino acid sequence in which 1 to 10, more preferably 1 to 5 amino acids are deleted, substituted, or added in comparison to a reference amino acid sequence.

As used herein, “genomic DNA” means DNA containing introns and “cDNA” means DNA containing no introns. For example, the base sequence of SEQ ID NO: 1 shows the base sequence of genomic DNA of a β-glucan-deficient gene; positions 328 to 1,954 and positions 2,699 to 3,367 of this base sequence are introns. Introns are removed by splicing in the process that the genomic DNA is transcribed into mRNA and translated into a protein in a host. Here, the host is preferably a plant and more preferably a barley. For example, SEQ ID NO: 3 shows a base sequence of cDNA of a β-glucan-deficient gene in which an intron portion is removed from the base sequence of SEQ ID NO: 1. As used herein, a “β-glucan-deficient gene” may mean both of the genomic DNA and the cDNA.

As used herein, a “β-glucan synthesis activity” means an activity participating in the pathway of β-glucan synthesis and promoting the synthesis of β-glucan. In addition, “β-glucan synthesis gene” means a gene encoding a protein having the function of participating in the pathway of β-glucan synthesis and promoting the synthesis of β-glucan. In one aspect, the present invention provides a method for breeding a barley having a genomic DNA of a β-glucan-deficient gene, comprising a selection step of selecting the barley by determining the barley as having a genomic DNA of a β-glucan-deficient gene when the base corresponding to position 4,275 of the genomic DNA consisting of the base sequence of SEQ ID NO: 4 located near the centromere of chromosome 7H of the barley is mutated from G to A. In the selection step according to the breeding method, the method for detecting the mutation of the genomic DNA is not particularly limited. For example, the region on genomic DNA to be detected may be subjected to PCR amplification, followed by determining the base sequence of the PCR fragment by the direct sequencing thereof or by sequencing after inserting the PCR fragment into a sequencing vector to determine whether the fragment has the intended mutation. However, because of simpler determination operation, it is preferable to detect the presence of the intended mutation using the cleavability by a restriction enzyme as an indicator.

Specifically, using a genomic DNA extracted from a barley as a template, for example, the DNA fragment consisting of the base sequence corresponding to positions 3,893 to 4,361 of the base sequence of SEQ ID NO: 4 is PCR amplified; the amplified 469 bp DNA fragment is cleaved with a restriction enzyme, BanI; the cleaved DNA fragment is detected by agarose electrophoresis or the like; and the barley can be determined as having a genomic DNA of a β-glucan-deficient gene when the DNA fragment is not cleaved with the restriction enzyme BanI. This method enables rapid determination when the base corresponding to position 4,275 of the genomic DNA consisting of the base sequence of SEQ ID NO: 4 is mutated to A. In addition, it similarly enables determination when the base corresponding to the position 4,275 is mutated to C or T.

TaqI, NlaIV, or the like as well as BanI can be used as a restriction enzyme. The recognition sequence of TaqI is 5′-TCGA-3′, which is cleaved into the form of 5′-T/CGA-3′ by digestion with TaqI. Thus, the base corresponding to the position 4,275 of the genomic DNA consisting of the base sequence of SEQ ID NO: 4 is cleaved when it has been mutated from G to A. The recognition sequence of NlaIV is 5′-GGNNCC-3′, which is cleaved into the form of 5′-GGN/NCC-3′ by digestion with NlaIV. Here, N means A, T, G, or C. Thus, the base corresponding to the position 4,275 of the genomic DNA consisting of the base sequence of SEQ ID NO: 4 is cleaved when it has been mutated from G to A, C, or T.

A plurality of the recognition sequences cleaved by restriction enzymes such as BanI, TaqI or NlaIV are present around the base corresponding to the position 4,275 of the genomic DNA consisting of the base sequence of SEQ ID NO: 4. In determining the mutation of the base corresponding to the 4,275 position by cleavage using each of these restriction enzymes, those skilled in the art can easily select which region of the base sequence of SEQ ID NO: 4 can be amplified to prepare a DNA fragment for determination. Usable restriction enzymes other than these restriction enzymes will be apparent to those skilled in the art.

The method for extracting a genomic DNA from a barley is not particularly limited; for example, the DNA can be extracted by collecting about 0.1 g of young leaves and milling them on a mortar after adding liquid nitrogen, followed by extraction using DNeasy Plant Mini Kit (Qiagen Inc.) according to an instruction.

The breeding method of the present embodiment can be carried out as follows, for example. A wild-type barley is first treated with a mutation-inducing agent such as ethyl methanesulfonate (EMS) to provide mutants. Subsequently, a genomic DNA is extracted from each of the resultant mutants, and the mutation of the base corresponding to the position 4,275 of the genomic DNA consisting of the base sequence of SEQ ID NO: 4 is detected by the above method to determine and select a barley individual having genomic DNA of a β-glucan-deficient gene. In this way, a barley having a genomic DNA of a β-glucan-deficient gene can be bred. Alternatively, a barley having a genomic DNA of a β-glucan-deficient gene can be bred by crossing a wild-type barley variety with a barley variety already shown to have a genomic DNA of a β-glucan-deficient gene and, from among the progenies thereof, determining and selecting a barley having the genomic DNA of a β-glucan-deficient gene by the same method as that described above. Alternatively, a barley having a genomic DNA of a β-glucan-deficient gene can be bred by artificially mutating the base corresponding to the position 4,275 of the genomic DNA consisting of the base sequence of SEQ ID NO: 4 in a wild type barley from G to A, C, or T using a molecular biological technique such as a gene-targeting approach by homologous recombination. The artificially introduced mutation is preferably mutation from G to A.

In another aspect, the present invention provides a method for breeding a barley having a genomic DNA of a β-glucan-deficient gene, comprising a selection step of selecting the barley by determining the barley as having a genomic DNA of a β-glucan-deficient gene when the base corresponding to position 2,385 of the genomic DNA consisting of the base sequence of SEQ ID NO: 4 located near the centromere of chromosome 7H of the barley is mutated from G to A. In the selection step according to the breeding method, the method for detecting the mutation of the genomic DNA is not particularly limited. For example, the region on a genomic DNA to be detected may be subjected to PCR amplification, followed by determining the base sequence of the PCR fragment by the direct sequencing thereof or by sequencing after inserting the PCR fragment into a sequencing vector to determine whether the fragment has the intended mutation. However, because of simpler determination operation, it is preferable to detect the presence of the intended mutation using the cleavability by a restriction enzyme as an indicator.

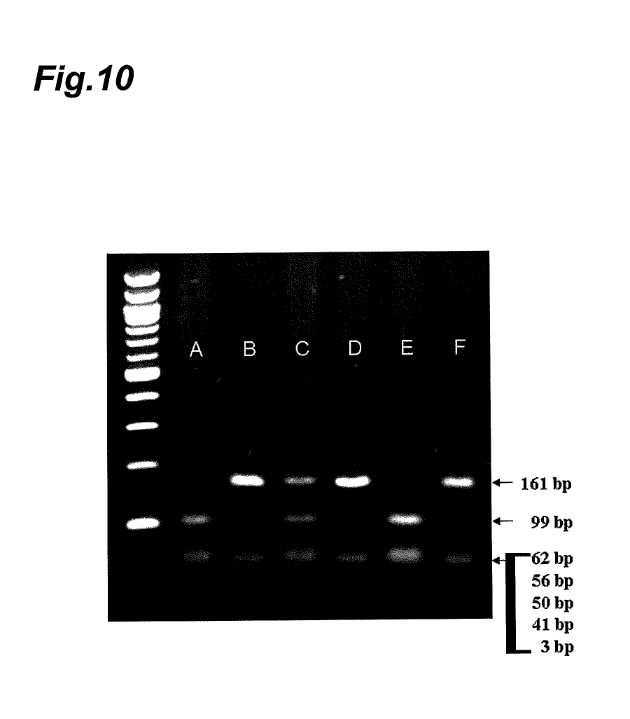

Specifically, using a genomic DNA extracted from a barley as a template, for example, the DNA fragment consisting of the base sequence corresponding to positions 2,219 to 2,529 of the base sequence of SEQ ID NO: 4 is PCR amplified, and the amplified 311 bp DNA fragment is cleaved with a restriction enzyme, Fnu4HI; the cleaved DNA fragment is detected by agarose electrophoresis or the like. In a wild-type variety, digestion with the restriction enzyme Fnu4HI cleaves the amplified fragment into 6 fragments with 3, 41, 50, 56, 62 and 99 bp. In contrast, when the base corresponding to position 2,385 of the genomic DNA consisting of the base sequence of SEQ ID NO: 4 is mutated from G to A or mutated from G to C or T, digestion with Fnu4HI cleaves the amplified fragment into 5 fragments with 3, 41, 50, 56 and 161 bp because one of the recognition sites of Fnu4HI is eliminated by single base substitution. The difference in the fragments produced by Fnu4HI digestion can be easily distinguished by electrophoresis or the like as shown in FIG. 10. This method enables rapid determination of the presence of mutation of the base corresponding to position 2,385 of the genomic DNA consisting of the base sequence of SEQ ID NO: 4.

The recognition sequence of Fnu4HI is 5′-GCNGC-3′, which is cleaved into the form of 5′-GC/NGC-3′ by Fnu4HI digestion. Here, N means A, T, G, or C. BisI, BsoFI, Fsp4HI, ItaI, SatI, and the like are known as restriction enzymes recognizing the same base sequence as that for Fnu4HI and cleaving the sequence into the same form as that for Fnu4HI. These restriction enzymes can be used in the same way as that for Fnu4HI.

A plurality of the recognition sequences cleaved by Fnu4HI are present around the base corresponding to the position 2,385 of the genomic DNA consisting of the base sequence of SEQ ID NO: 4. In determining the mutation of the base corresponding to the 2,385 position by cleavage using Fnu4HI, those skilled in the art can easily select which region of the base sequence of SEQ ID NO: 4 can be amplified to prepare a DNA fragment for determination. Usable restriction enzymes other than Fnu4HI will be apparent to those skilled in the art.

The breeding method of the present embodiment can be carried out as follows, for example. A wild-type barley is first treated with a mutation-inducing agent such as ethyl methanesulfonate (EMS) to provide mutants. Subsequently, a genomic DNA is extracted from each of the resultant mutants, and the mutation of the base corresponding to the position 2,385 of the genomic DNA consisting of the base sequence of SEQ ID NO: 4 by the above method is detected to determine and select a barley individual having a genomic DNA of a β-glucan-deficient gene. In this way, a barley having a genomic DNA of a β-glucan-deficient gene can be bred. Alternatively, a barley having genomic DNA of a β-glucan-deficient gene can be bred by crossing a wild-type barley variety with a barley variety already shown to have a genomic DNA of a β-glucan-deficient gene and, from among the progenies thereof, determining and selecting a barley having the genomic DNA of a β-glucan-deficient gene by the same method as that described above. Alternatively, a barley having a genomic DNA of a β-glucan-deficient gene can be bred by artificially mutating the base corresponding to the position 2,385 of the genomic DNA consisting of the base sequence of SEQ ID NO: 4 in a wild type barley from G to A, C, or T using a molecular biological technique such as a gene-targeting approach by homologous recombination. The artificially introduced mutation is preferably mutation from G to A.

In one aspect, the present invention provides a genomic DNA and a cDNA of a β-glucan synthesis gene in a barley. It is possible to develop a plant variety showing a high production level of β-glucan by a method such as a method involving introducing the genomic DNA or cDNA of a β-glucan synthesis gene of the present invention into the genome of a plant such as a barley and increasing the number of copies thereof. Alternatively, it is also possible to industrially produce β-glucan on a large scale by introducing the genomic DNA or cDNA of a β-glucan synthesis gene of the present invention into a microorganism including a prokaryote such as Escherichia coli or a eukaryote such as yeast. β-Glucan has a high use value because it has physiological effects such as the suppression of the increased blood glucose level and the reduction of blood cholesterol and effects such as the promotion of immune function.

When a β-glucan synthesis gene is introduced into a plant body, a β-glucan synthesis gene expression vector is first constructed. The expression vector comprises a promoter expressible in a plant body, a β-glucan synthesis gene, and a translational termination sequence. The β-glucan synthesis gene may be a genomic DNA or a cDNA. Here, the plant body providing a host is not particularly limited; however, it is preferably a barley. The expression vector may also comprise a replication origin in E. coli, a selection marker in E. coli (an ampicillin-resistant gene, a tetracycline resistance gene, or the like), a selection marker in a plant body (a kanamycin resistance gene, a hygromycin resistance gene, a bialaphos (bar) resistance gene, or the like), a polyadenylation sequence, an enhancer sequence, and the like.

The promoter expressible in a plant body may be one inducing seed-specific expression or one performing constitutive expression independent of tissue. Examples thereof can include an ubiquitin promoter, an actin promoter, an Em promoter, and the 35S promoter of cauliflower mosaic virus.

The introduction of a gene into a barley can be performed using a common particle-bombardment method or an Agrobacterium method. For example, a gene is introduced by delivering gold particles coated with a β-glucan synthesis gene expression vector DNA into an immature embryo taken out of an immature barley seed using the particle-bombardment method. Subsequently, the gene-introduced immature embryo can be cultured in a medium containing auxin or the like as a phytohormone, followed by decreasing the concentration of auxin to redifferentiate the individual to provide a transformant plant body. For example, when a β-glucan synthesis gene expression vector containing a biapholas resistance gene has been introduced into a barley, the transformant plant body having incorporated the transgene can be selected using a medium containing biapholas because it becomes resistant to biapholas. Alternatively, a β-glucan synthesis gene can be introduced into a barley by immersing a barley-derived callus in a solution of Agrobacterium having a β-glucan synthesis gene expression vector introduced thereinto for infection with Agrobacterium. For example, when a β-glucan synthesis gene expression vector containing a hygromycin resistance gene has been introduced into a barley, the transformant having incorporated the gene can be selected in a medium containing hygromycin and a bacteria-eliminating agent such as carbenicillin for eliminating Agrobacterium. Thereafter, the resultant transformant can be cultured in an auxin-removed redifferentiation medium for redifferentiation and then turned into a rooting medium to provide a transformant plant body holding the β-glucan synthesis gene so as to be expressible.

A microorganism exemplified by a prokaryote such as E. coli or a eukaryote such as yeast can also be used as a host for introducing the β-glucan synthesis gene expression vector. By way of non-limited example, the K-12 strain is preferably used as E. coli and a pBR322 or pUC plasmid is typically used as a vector. A tryptophan (trp) promoter, a lactose (lac) promoter, or the like can be used as a promoter for E. coli. As yeast, for example, a Saccharomyces yeast, e.g., Saccharomyces cerevisiae, a bakery yeast, or Pichia pastoris, a petroleum yeast, can be used. As a promoter for yeast, for example, a promoter for alcohol dehydrogenase gene, a promoter for acid phosphatase gene, or the like can be used.

In one aspect, the present invention provides a method for decreasing or abolishing β-glucan in barley kernels, comprising a step of suppressing the expression of genomic DNA of a β-glucan synthesis gene as DNA consisting of a base sequence having 90% or more homology to the base sequence of SEQ ID NO: 4 and producing a protein having a β-glucan synthesis activity when transcribed and translated or genomic DNA of a β-glucan synthesis gene consisting of the base sequence of SEQ ID NO: 4.

In one aspect, the present invention provides a method for decreasing or abolishing β-glucan in barley kernels, comprising a step of suppressing the expression of genomic DNA of a β-glucan synthesis gene as DNA consisting of a base sequence having 90% or more homology to the base sequence of SEQ ID NO: 15 and producing a protein having a β-glucan synthesis activity when transcribed and translated or genomic DNA of a β-glucan synthesis gene consisting of the base sequence of SEQ ID NO: 15.

A known method involving introducing an siRNA or antisense nucleic acid into cells can be used as a method for suppressing the expression of DNA. Specifically, for example, a vector for expressing an siRNA targeting the genomic DNA of a β-glucan synthesis gene or a vector for expressing an antisense RNA targeting the genomic DNA of a β-glucan synthesis gene may be introduced into a barley using a particle-bombardment method or an Agrobacterium method as described above. According to this method, a barley decreasing or completely deficient in β-glucan in kernels can be obtained by suppressing the expression of the genomic DNA of a β-glucan synthesis gene.

### EXAMPLES

The present invention will be described more specifically below with reference to Examples of the present invention. However, the present invention is not intended to be limited to these Examples, and various changes can be made without departing from the technical idea of the present invention.

(Production of Barley Cultivar “Nishinohoshi” Near-Isogenic Line)

The present inventors have found that a barley cultivar (line), OUM125, is deficient in β-glucan, and identified a gene deficient in β-glucan based thereon, thereby accomplishing the present invention. OUM125 is a semidwarf mutant produced by treating a six-rowed naked barley cultivar, Akashinriki, with ethyl methanesulfonate (EMS) in Okayama University. The seeds of OUM125 used in this Example were provided by Dr. Kazuhiro Sato in the Barley and Wild Plant Resource Center, Institute of Plant Science and Resources, Okayama University.

Nishinohoshi is a two-rowed barley variety having superior traits. Izumi-kei A41 is a line produced by crossing a progeny between Saikaikawa 55 and OUM125 with Saikaikawa 54 (development name of Nishinohoshi) in the Kyushu Agricultural Experiment Station in 1997, and has β-glucan deficiency and a naked caryopsis. Inventors used Izumi-kei A41 as a nonrecurrent parent and Nishinohoshi as a recurrent parent for crossing to produce a β-glucan-deficient and hulled Nishinohoshi near-isogenic line.

(Analysis of β-Glucan Deficiency)

Individual kernels are each cut in the transverse direction and a portion away from the embryo was crushed using a pair of pinchers. The β-glucan deficiency was determined by treating the crushed kernels with lichenase and β-glucosidase and color developing glucose using a glucose assay kit (Glucose C-II Test, Wako Pure Chemical Industries, Ltd.). The reaction solution became colorless or light pink for β-glucan-deficient kernels, whereas the reaction solution became deep red for wild-type kernels.

(Linkage Analysis of β-Glucan Deficiency and Hulled and Naked Caryopsis)

The linkage analysis of β-glucan deficiency and naked caryopsis was performed in 228 individuals obtained by crossing Nishinohoshi with its near-isogenic line. The results of the linkage analysis are shown in Table 1. The segregation ratio of β-glucan-deficient type versus wild type was 1:3. Thus, β-glucan deficiency was probably due to a monofactorial recessive gene. This gene was designated a β-glucan-deficient gene ((1-3,1-4)-β-D-glucanless, bgl). A significant linkage was detected between a β-glucan-deficient gene and a naked caryopsis (nud) gene. The recombination value between these genes was calculated to be 14.4%±2.5% by the maximum-likelihood method of Allard (Hilgardia 24:235-278, 1956). The nud gene is known to be present on the long arm of chromosome 7H; thus, the β-glucan-deficient gene was also considered to be located on the chromosome 7H.

TABLE 1
Joint Segregation of β-Glucan Deficiency and Naked Caryopsis
in F₂ of Nishinohoshi (β-Glucan Deficient + Naked) Near-Isogenic Line ×
Nishinohoshi

Presence
of β-Glucan

| | Normal | Deficient | Total |
|---|---|---|---|
| Hulled | 153 | 15 | 168 |
| Naked | 16 | 44 | 60 |
| Total | 169 | 59 | 228 |

Test of Segregation Ratio for Normal Type and Deficient Type of β-Glucan
X²(3:1) = 0.0936, 0.90 > p > 0.75
Test of Independence between β-Glucan Deficiency and Naked Caryopsis
X²L = 101.3, p < 0.01
Recombination Value between β-Glucan Deficiency and Naked Caryopsis = 14.4 ± 2.5%

(Mapping)

Genomic DNAs were extracted from Nishinohoshi, its near-isogenic line, and 228 individuals obtained by their crossing by the CTAB method. Using the SSR marker reported by Ramsay et al. (Genetics 156:1997-2005, 2000), the genomic DNAs were analyzed and the β-glucan-deficient gene was mapped. The SSR markers on chromosome 7H showing polymorphism between the parents were selected and PCR analyzed. The position of each marker on the chromosome was determined by analysis using a wheat line having a barley chromosome added (Islam, Sakamoto, S. ed., Proc. 6th Int. Wheat Genet. Symp. Maruzen Kyoto. pp. 233-238, 1983). The recombination value was calculated using MAPMAKER version 2.0 (Lander et al., Genomics 1: 174-181, 1987). The genetic distance was calculated using Kosambi function (Kosambi, Ann. Eugen. 12: 172-175, 1944).

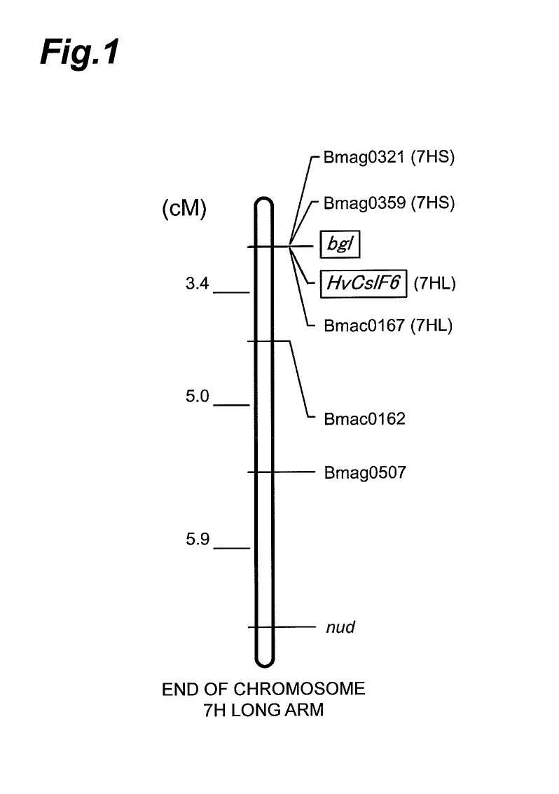

The results of mapping are shown in FIG. 1. The β-glucan-deficient gene was mapped at a position 3.4 cM distant from Bmac0162, indicating that it is co-localized with Bmag0321, Bmag0359, Bmac0167 and HvCslF6 genes. The HvCslF6 gene is one of the cellulose synthase-like gene subfamilies. The β-glucan-deficient gene was demonstrated to be located near the centromere of chromosome 7H.

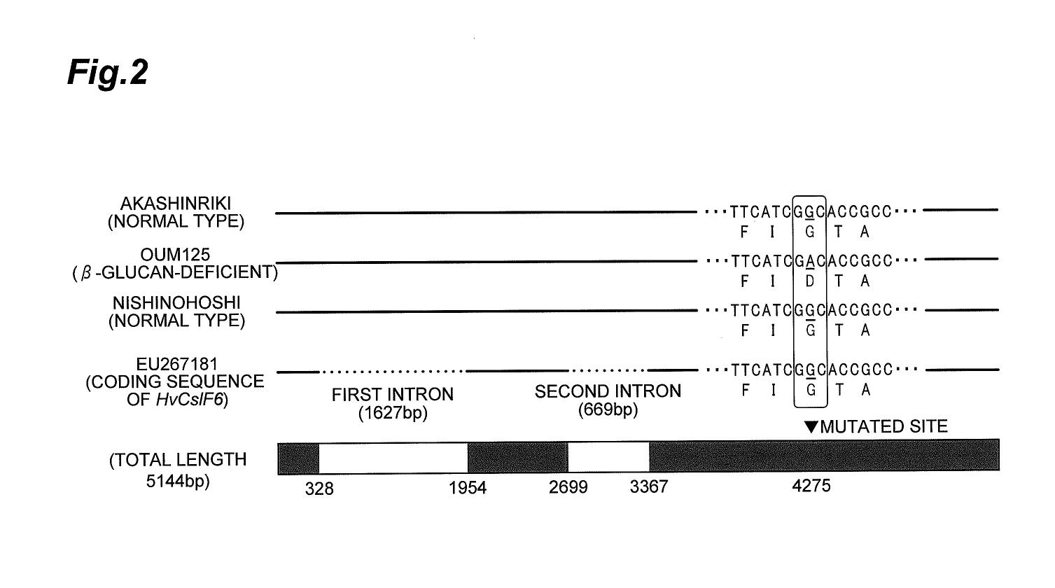

It was further demonstrated that a single nucleotide polymorphism (SNP) capable of being detected with the restriction enzyme BanI is present in HvCslF6 gene and the phenotype of β-glucan deficiency completely agrees with the genotype of HvCslF6 gene locus. That is, as shown in FIG. 2, HvCslF6 gene whose base of position 4,275 is substituted from G to A is a β-glucan-deficient gene; when the β-glucan-deficient gene is held in homozygous form, kernels are completely deficient in β-glucan. As shown in FIG. 2, the deduced amino acid is glycine for the wild-type variety and aspartic acid for the variety having the β-glucan-deficient gene.

(CAPS Analysis)

The presence of a β-glucan-deficient gene can be clearly determined by a co-dominant CAPS (cleaved amplified polymorphic sequence) marker. Here, the co-dominant marker means a marker by which a heterotype pattern can be distinguished from both homotypes of parents. To perform the CAPS analysis of a β-glucan-deficient gene, the primers (CAPS marker): 5′-GCCAAGACCAAGTACGAGAAGC-3′ (forward, SEQ ID NO: 11) and 5′-TGTTCTTGGAGAAGAAGATCTCG-3′ (reverse, SEQ ID NO: 12) were prepared.

Using these primers, a genomic DNA of a barley is subjected to PCR to provide an amplified fragment with 469 bp. For a wild type variety, the amplified fragment is cleaved into 382 bp and 87 bp by digestion with the restriction enzyme BanI. In contrast, for a variety having a β-glucan-deficient gene, the amplified fragment is not cleaved by digestion with the restriction enzyme BanI because of the absence of a BanI recognition site due to single base substitution. The recognition sequence of BanI is 5′-GGYRCC-3′, which is cleaved into the form of 5′-G/GYRCC-3′ by digestion with BanI. Here, Y means C or T, and R means A or G.

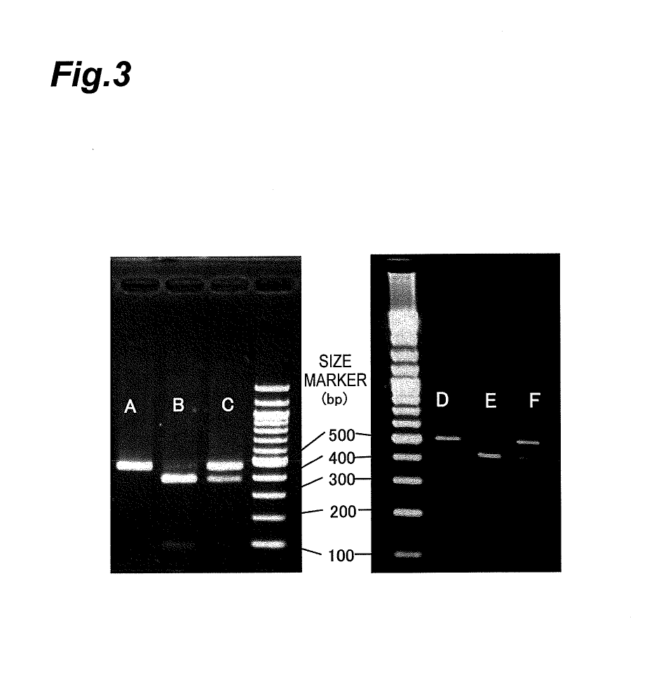

FIG. 3 shows the results of determining the presence of a β-glucan-deficient gene by the CAPS analysis. After the PCR amplification of the genomic DNA using the above primers, the amplified fragment was cleaved with the restriction enzyme BanI and subjected to agarose gel electrophoresis. Lanes A to F are the results of analyzing the genomic DNA of OUM125 (β-glucan deficient), Akashinriki (wild type), F1 (hetero type) between OUM125 and Akashinriki, a Nishinohoshi near-isogenic line (β-glucan-deficient and naked), Nishinohoshi (wild type), and F1 (hetero type) between the Nishinohoshi near-isogenic line (β-glucan-deficient and naked) and Nishinohoshi.

(Measurement of Content of Starch, β-Glucan and Arabinoxylan)

Nishinohoshi (bgl) and Nishinohoshi were grown and harvested. Here, Nishinohoshi (bgl) means a Nishinohoshi near-isogenic line which is β-glucan-deficient and hulled. After the harvest, they were each ground and passed through a 0.5 mm sieve, followed by measuring the contents of β-glucan and arabinoxylan in a mature whole-kernel sample. The content of β-glucan was measured by an enzymatic method (McCleary and Codd, 1991) using Mixed Linkage β-Glucan Assay Kit (Megazyme International, Ireland). The content of arabinoxylan was determined by, according to the method of Sekiwa et al. (2003), hydrolyzing the sample with sulfuric acid and then separately measuring the contents of arabinose and xylose. The content of arabinose was measured by an enzymatic method as described in “Arabinan assay procedure (Megazyme International Ireland 2002)”. The content of xylose was measured using D-Xylose Assay Kit (Megazyme International, Ireland). Before measuring the contents of β-glucan and arabinoxylan, free low molecular saccharides in the ground sample was removed by treatment with 80% ethanol. The results are shown in Table 2.

β-Glucan was not detected in a Nishinohoshi (bgl) kernel, while 3.2% by mass of β-glucan was found to be contained in a Nishinohoshi kernel. The content of arabinoxylan was 6.3% by mass in the Nishinohoshi kernel, while being 7.2% by mass in the Nishinohoshi (bgl) kernel.

TABLE 2
Content of β-Glucan and Arabinoxylan in Kernel of
Nishinohoshi (bgl) Near-Isogenic Line

| | β-Glucan | Arabinoxylan |
|---|---|---|
| Variety/Line | (%) | (%) |
| Nishinohoshi (bgl) Near-Isogenic Line | 0.0a | 7.2b |
| Nishinohoshi | 3.2b | 6.3a |

Significant difference are present at a 5% level between the numerical values to which the different alphabets are attached (Fisher's PLSD test).

(Observation Under Light Microscope)

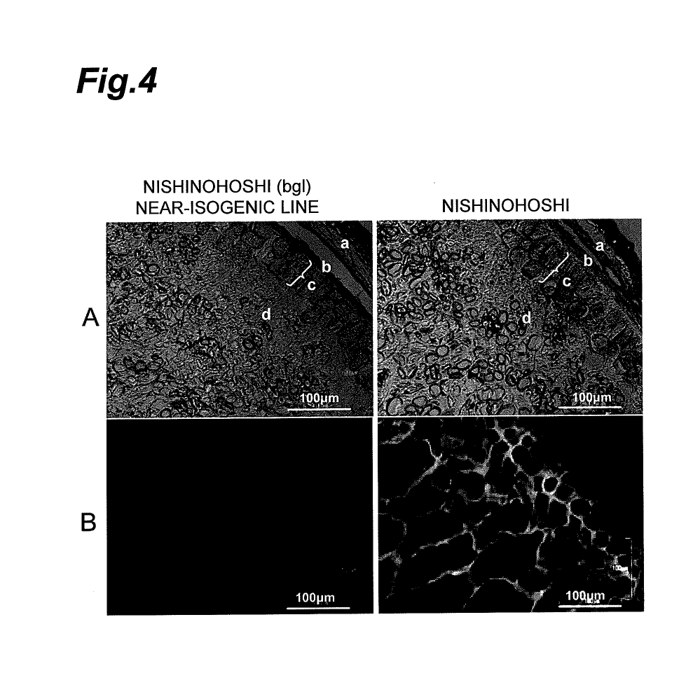

Nishinohoshi (bgl) and Nishinohoshi kernels were each sliced, fixed in glutaraldehyde, dehydrated by a series of ethanol treatments, and embedded in a low temperature polymerization resin, Technovit 7100 (Heraeus Kulzer, Germany). A 7 μm-thick section was double-stained using 0.5% Fast Green FCF (Sigma-Aldrich Corporation, USA) and 0.01% Calcofluor (Wako Pure Chemical Industries, Ltd.). The section was observed by a factor of 200 times under a light microscope equipped with a UV filter (Axiophot, Carl Zeiss, Germany). The cell wall, cytoplasm and nucleus are stained by Fast Green FCF, and β-glucan is stained by Calcofluor. The results are shown in FIG. 4. The observation magnification was 200 times. In FIG. 4, A indicates bright-field observation images; B indicates ultraviolet radiation images. In FIG. 4, a means a husk; b, a seed coat and a seed vessel; c, an aleurone layer; and d, an endosperm. Fluorescence was observed in the aleurone and endosperm cell wall for the sample of Nishinohoshi, while their fluorescence was not observed for Nishinohoshi (bgl). This result shows that the line having a β-glucan-deficient gene is completely deficient in β-glucan in both of the aleurone layer and the endosperm.

(Observation Under Electron Microscope)

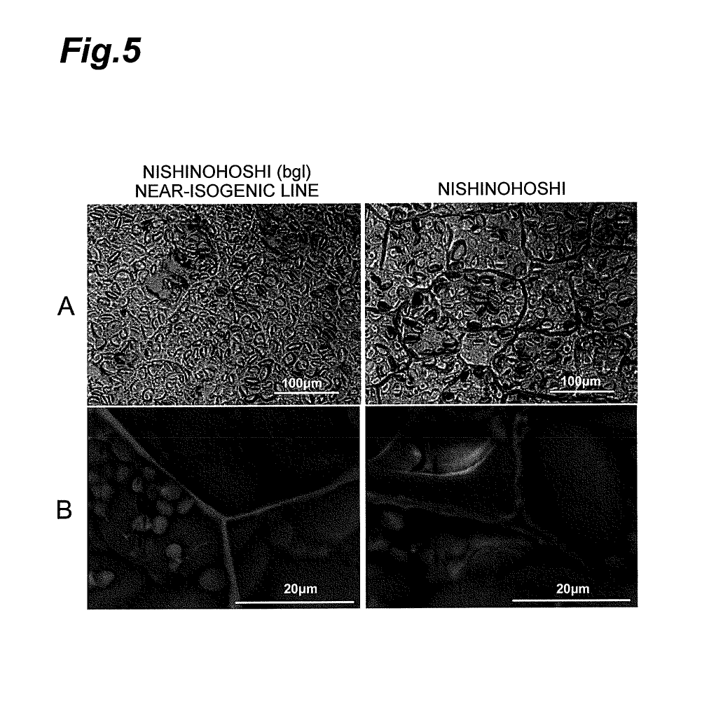

Horizontal sections of Nishinohoshi (bgl) and Nishinohoshi kernels were each observed by a factor of 5,000 times under a scanning electron microscope (N-3400, Hitachi). The results are shown in FIG. 5. In FIG. 5, A indicates bright-field observation images (observation magnification: 200 times) under a light microscope; B indicates electron microscope observation images (observation magnification: 5,000 times). It was demonstrated that whereas Nishinohoshi had a thick endosperm cell wall, Nishinohoshi (bgl) had a markedly thin endosperm cell wall. The cell wall of aleurone cells was not found to be different in thickness therebetween.

(Measurement of β-Glucan Content in Seedling Leaf)

Before the extension of the stem of Nishinohoshi (bgl) and Nishinohoshi, their infant plants in a rosette state were each collected from a field and freeze dried. Subsequently, the contents of β-glucan in the leaf blade and sheath thereof were each measured using Mixed Linkage β-Glucan Assay Kit (Megazyme International, Ireland). Before the measurement of the β-glucan content, free low molecular saccharides were removed by 80% hot ethanol treatment. The results are shown in Table 3.

Little or no β-glucan was detected in leaves of Nishinohoshi (bgl), while 13.5 mg/g of β-glucan was contained in leaves of Nishinohoshi. The above result shows that Nishinohoshi (bgl) is deficient in β-glucan not only in kernels but also in the vegetative organ.

TABLE 3
Content of β-Glucan in Seedling Leaf of Nishinohoshi (bgl)
Near-Isogenic Line

| Variety/Line | β-Glucan (mg/g) |
|---|---|
| Nishinohoshi (bgl) Near-Isogenic Line | 0.1a |
| Nishinohoshi | 13.5b |

Significant difference are present at a 5% level between the numerical values to which the different alphabets are attached (Fisher's PLSD test).

(Measurement of Kernel Hardness)

The kernel hardness of Nishinohoshi (bgl) and Nishinohoshi kernels were each measured. The kernel hardness was measured in 300 grains each of the kernels using a single-kernel hardness tester (SKCS-4100, Perten Inc., Sweden). The milling time is set at the time necessary for milling 180 g of kernels (raw barley) to a yield of 56% using a test mill (TM-05, Satake Corporation, Higashi-Hiroshima City), and the kernel breakage rate was determined as the mass ratio of broken kernels and defective kernels in 10 g of the milled barley. The results are shown in Table 4.

Nishinohoshi (bgl) kernels were shown to become markedly soft and brittle compared to Nishinohoshi kernels. This result probably stems from the thinning of the endosperm cell wall due to β-glucan deficiency. The characteristic that the kernels are soft and brittle has the usefulness that it makes the crushing thereof easy in the production of an animal feed and facilitates the processing thereof into grits.

TABLE 4
Characteristics of Kernel of Nishinohoshi (bgl) Near-Isogenic Line

| | Kernel | | |
|---|---|---|---|
| | Hardness | Milling | Kernel |
| | (Hardness | Time | Breakage |
| Variety/Line | Index) | (second) | Rate (%) |
| Nishinohoshi (bgl) | 30.8a | 265a | 35.9b |
| Near-Isogenic Line | | | |
| Nishinohoshi | 74.9b | 497b | 3.8a |

Significant difference are present at a 5% level between the numerical values to which the different alphabets are attached (Fisher's PLSD test).

(Polymorphism in Promoter Region of HvCslF6 Gene)

Barley lines, TR251 and CDC-Bold, are known to be different in the production amount of β-glucan (Journal of Cereal Science 48:647-655, 2008). TR251 is a high β-glucan production line, and CDC-Bold is a low β-glucan production line. The present inventors have believed that the difference in the production amount of β-glucan between these lines may be due to the mutation of the base sequence of HvCslF6 gene, and determined the base sequences of the genomes of HvCslF6 gene regions of these barley lines. TR251 was distributed by Dr. W. G. Legge (Brandon Research Centre, Agriculture and Agri-Food Canada, Canada). CDC-Bold was distributed by Dr. B. G. Rossnagel (Crop Development Centre, University of Saskatchewan, Canada).

Genomic DNAs were extracted from these barley lines by the CTAB method, and the base sequences of the genomes were determined which lay about 2 kb upstream of the initiation codons of HvCslF6 genes, containing the promoter regions of HvCslF6 genes (hereinafter referred to as “promoter sequences”). The promoter sequences of the HvCslF6 genes of TR251 and CDC-Bold are shown in SEQ ID NOS: 13 and 14.

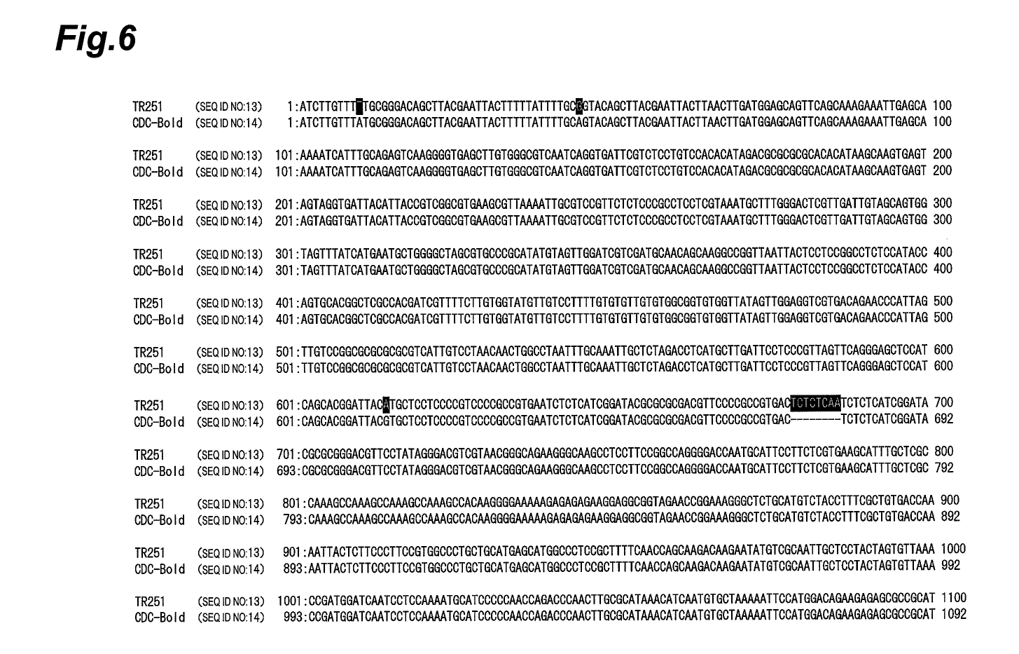

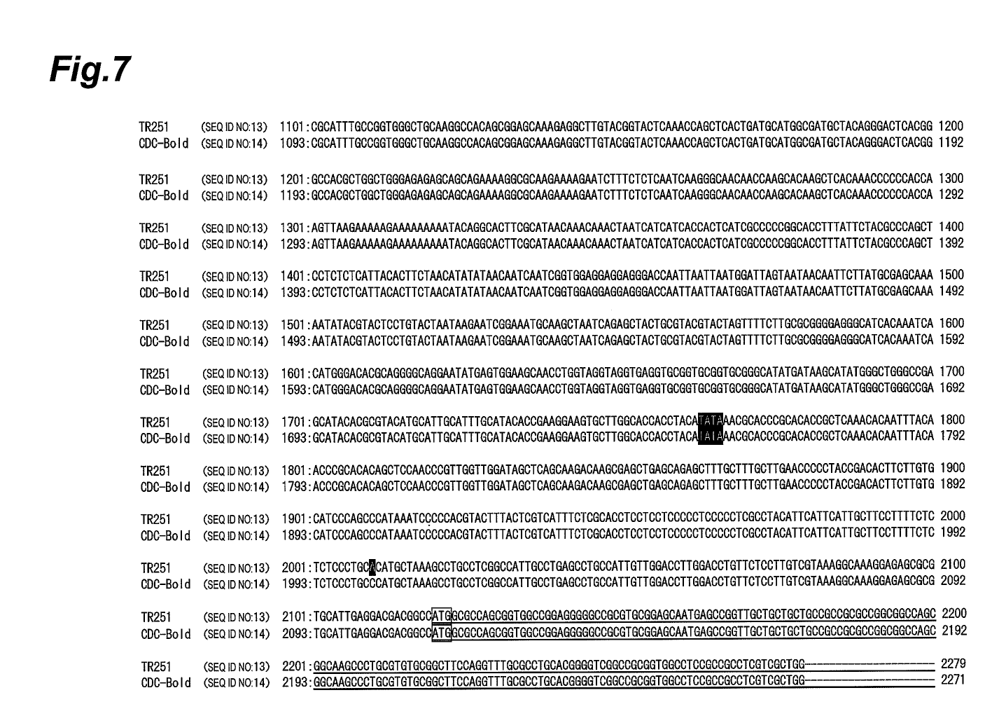

As shown in FIGS. 6 and 7, the comparison of these base sequences demonstrated that the bases corresponding to positions 10, 45, 614 and 2,002 of the base sequence of SEQ ID NO: 14 (the promoter sequence of CDC-Bold) in the promoter sequence of TR251 (SEQ ID NO: 13) were mutated from A to T, from A to G, from G to A, and from C to A, respectively. In addition, the 8 base pairs of 5′-TCTCTCAA-3′ were observed to be inserted between the bases corresponding to positions 678 and 679 of the base sequence of SEQ ID NO: 14. In the figure, each mutation site is indicated by an open character. It was shown that TR251 displayed the phenotype of highly producing β-glucan because of the differences in the base sequence.

A barley variety highly producing or lowly producing β-glucan can be efficiently bred using polymorphism in these promoter regions as an indicator.

Typical TATA boxes were found at positions 1,755 to 1,758 (about 350 bp upstream of the initiation codon of the HvCslF6 gene) of the base sequence of SEQ ID NO: 14 (the promoter sequence of CDC-Bold) and the positions corresponding thereto in the promoter sequence of TR251 (SEQ ID NO: 13). In FIG. 7, the TATA boxes are each indicated by open characters. The initiation codons of HvCslF6 genes were each boxed, and the coding regions were underlined.

(Polymorphism in Coding Region of HvCslF6 Gene)

The base sequence of the genomic DNA of the coding region of the HvCslF6 gene of TR251 is shown in SEQ ID NO: 23; the base sequence of the cDNA of the region, in SEQ ID NO: 24; and the deduced amino acid sequence therefrom, in SEQ ID NO: 25. The base sequence of the genomic DNA of the coding region of the HvCslF6 gene of CDC-Bold is shown in SEQ ID NO: 26; the base sequence of the cDNA of the region, in SEQ ID NO: 27; and the deduced amino acid sequence therefrom, in SEQ ID NO: 28. It was demonstrated that the bases corresponding to positions 174, 1,768 and 2,505 of the sequence of the cDNA of the HvCslF6 gene of Akashinriki (wild type) (SEQ ID NO: 6) in the base sequence of the cDNA of the HvCslF6 gene of TR251 (SEQ ID NO: 24) were mutated from G to A, from G to A, and from T to G, respectively. Of these mutations, the mutation in the base of the position 1,768 was demonstrated to result in the mutation of the amino acid corresponding to position 590 of the amino acid sequence of the HvCslF6 protein of Akashinriki (wild type) (SEQ ID NO: 5) in the amino acid sequence of the HvCslF6 protein of TR251 (SEQ ID NO: 25) from alanine to threonine These mutations also probably have the possibility of having to do with the fact that TR251 is a high β-glucan production line.

(Obtaining New β-Glucan-Deficient Mutant)

The inventors have discovered a new β-glucan-deficient mutant of a barley variety, Sachiho Golden (TN5) (wild type), and designated it as KM27. Then, the base sequences of the HvCslF6 genes of Sachiho Golden and KM27 were determined. The HvCslF6 gene of KM27 is hereinafter referred to as a β-glucan-deficient gene. The base sequence of the genome of the HvCslF6 gene of Sachiho Golden is shown in SEQ ID NO: 15; the base sequence of the cDNA of the gene, in SEQ ID NO: 16; and the deduced amino acid sequence therefrom, in SEQ ID NO: 17. The base sequence of the genome of the β-glucan-deficient gene of KM27 is shown in SEQ ID NO: 18; the base sequence of the cDNA of the gene, in SEQ ID NO: 19; and the deduced amino acid sequence therefrom, in SEQ ID NO: 20.

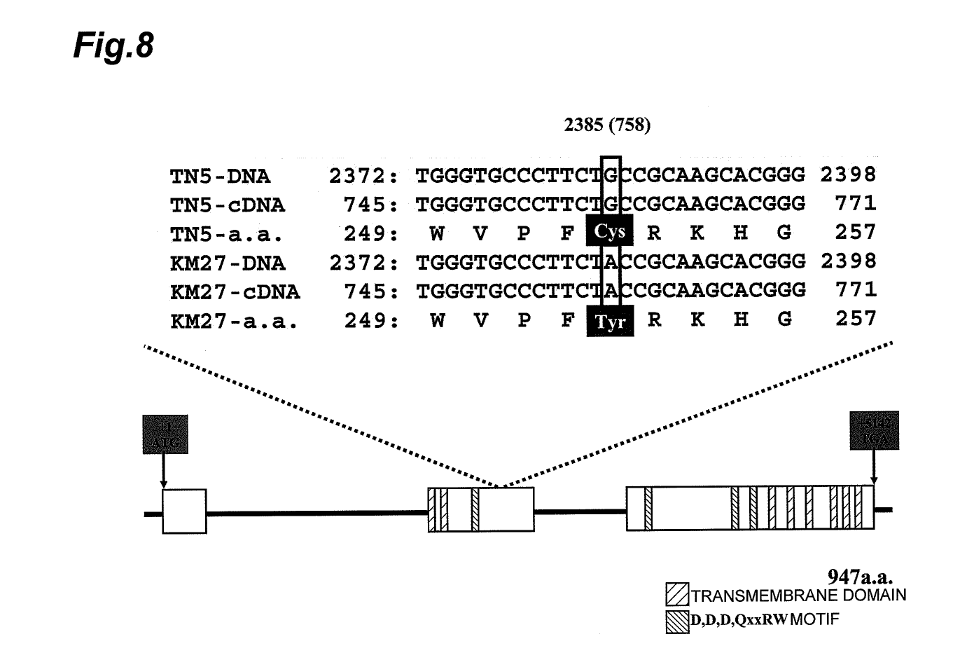

As shown in FIG. 8, it was demonstrated that the base of position 2,385 of the base sequence of the genome of the β-glucan-deficient gene of KM27 shown in SEQ ID NO: 18 is mutated from G as the base of the HvCslF6 gene of Sachiho Golden corresponding to the former base to A. The base of position 2,385 of SEQ ID NO: 18 corresponds to that of position 758 of SEQ ID NO: 19 (the base sequence of the cDNA of the β-glucan-deficient gene of KM27). This mutation was demonstrated to result in the mutation of the amino acid of position 253 of the amino acid sequence encoded by the β-glucan-deficient gene of KM27 from cysteine as the amino acid residue on HvCslF6 protein of Sachiho Golden, corresponding to the former amino acid to tyrosine. This mutation is different from the mutation identified in the β-glucan-deficient gene of each of OUM125 and Nishinohoshi (bgl).

The fact that two different mutants having mutations in HvCslF6 genes, having β-glucan deficiency were obtained is more strong evidence that the HvCslF6 gene is a β-glucan synthesis gene.

(Prediction of Three-Dimensional Structure of HvCslF6)

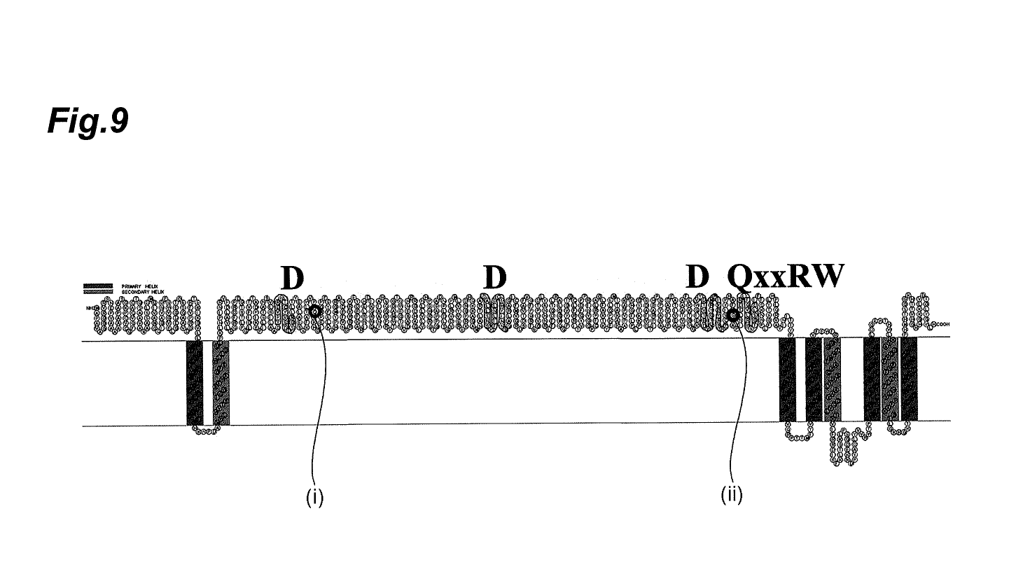

The three-dimensional structure of HvCslF6 protein was predicted using a software for predicting the three-dimensional structure of protein, SOSUI (freeware, http://bp.nuap.nagoya-u.ac.jp/sosui/). The results are shown in FIG. 9. In FIG. 9, the site in which cysteine is mutated to tyrosine in KM27 is indicated by (i), and the site in which glycine is mutated to aspartic acid in OUM125 is indicated by (ii). The positions of motif D and motif QxxRW are indicated by D and QxxRW, respectively.

(CAPS Analysis)

To perform the CAPS analysis of the same β-glucan-deficient gene as that of KM27, the following primers (CAPS marker) were prepared: 5′-ATCAAGGAGCCCATCCTCTC-3′ (HvCSLF6_MPP2-1F, SEQ ID NO: 21) and 5′-TTGATCCTGGCCTTGAACTC-3′ (HvCSLF6_MPP2-1R, SEQ ID NO: 22).

A Genomic DNA of a barley is subjected to PCR using these primers to provide an amplified fragment with 311 bp. For a wild-type variety, this fragment is cleaved into 6 fragments with 3, 41, 50, 56, 62 and 99 bp by digestion with the restriction enzyme Fnu4HI. In contrast, for a variety having the same β-glucan-deficient gene as that of KM27, digestion with Fnu4HI cleaves the above amplified fragment into 5 fragments with 3, 41, 50, 56 and 161 bp because the single base substitution eliminates one recognition site of Fnu4HI. The difference in these fragments produced by Fnu4HI digestion can be easily distinguished by electrophoresis or the like. The recognition sequence of Fnu4HI is 5′-GCNGC-3′, which is cleaved into the form of 5′-GC/NGC-3′ by Fnu4HI digestion. Here, N means A, T, G, or C. BisI, BsoFI, Fsp4HI, ItaI, SatI, and the like are known as restriction enzymes recognizing the same base sequence as that for Fnu4HI and cleaving the sequence into the same form as that for Fnu4HI. These restriction enzymes can be used in the same way as that for Fnu4HI.

FIG. 10 shows the results of determining the presence of the same β-glucan-deficient gene as that of KM27 by the CAPS analysis. Genomic DNAs were each PCR amplified using the above primers, and the amplified fragment was cleaved with the restriction enzyme Fnu4HI and subjected to agarose gel electrophoresis. Lanes A to F are the results of analyzing the genomic DNA of Sachiho Golden (wild type), KM27 (β-glucan-deficient), F1 (hetero type) between Sachiho Golden and KM27, KM27 (β-glucan-deficient), Nishinohoshi (bgl) (β-glucan-deficient), and F1 (hetero type) between Sachiho Golden and KM27.

### INDUSTRIAL APPLICABILITY

The use of the present invention enables the elucidation of a gene involved in the synthesis of β-glucan in a plant in the field of life science. If the gene involved in the synthesis of β-glucan is elucidated, it is expected that the results can be used to genetically control the content of β-glucan in breeding. The use thereof also enables the breeding of a barley variety for brewing/animal consumption containing no β-glucan in the agricultural field. Its application to a food using the functionality of arabinoxylan can also be carried out.

1-16. (canceled)

17. An isolated DNA selected from the group consisting of the DNAs of (a) to (t):
(a) a DNA consisting of a base sequence having 90% or more homology to the base sequence of SEQ ID NO: 1 and producing a protein lacking a β-glucan synthesis activity when transcribed and translated;
(b) a DNA consisting of a base sequence having 90% or more homology to the base sequence of SEQ ID NO: 1 and producing a protein lacking a β-glucan synthesis activity when transcribed and translated, wherein the protein consists of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is an amino acid other than glycine;
(c) a DNA consisting of a base sequence having 90% or more homology to the base sequence of SEQ ID NO: 1 and producing a protein lacking a β-glucan synthesis activity when transcribed and translated, wherein the protein consists of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is aspartic acid;
(d) a DNA consisting of the base sequence of SEQ ID NO: 1;
(e) a DNA consisting of a base sequence having 90% or more homology to the base sequence of SEQ ID NO: 18 and producing a protein lacking a β-glucan synthesis activity when transcribed and translated;
(f) a DNA consisting of a base sequence having 90% or more homology to the base sequence of SEQ ID NO: 18 and producing a protein lacking a β-glucan synthesis activity when transcribed and translated, wherein the protein consists of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is an amino acid other than cysteine;
(g) a DNA consisting of a base sequence having 90% or more homology to the base sequence of SEQ ID NO: 18 and producing a protein lacking a β-glucan synthesis activity when transcribed and translated, wherein the protein consists of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is tyrosine;
(h) a DNA consisting of the base sequence of SEQ ID NO: 18;
(i) a DNA encoding a protein lacking a β-glucan synthesis activity, wherein the protein consists of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is an amino acid other than glycine;
(j) a DNA encoding a protein lacking a β-glucan synthesis activity, wherein the protein consists of an amino acid sequence in which one or several amino acids are deleted, substituted, or added in the amino acid sequence of SEQ ID NO: 2, wherein the amino acid corresponding to position 660 of the amino acid sequence of SEQ ID NO: 2 is aspartic acid;
(k) a DNA consisting of the base sequence of SEQ ID NO: 3;
(l) a DNA encoding a protein lacking a β-glucan synthesis activity, wherein the protein consists of an amino acid sequence in which one or several amino acids are deleted, substituted, or added in the amino acid sequence of SEQ ID NO: 20, wherein the amino acid corresponding to position 253 of the amino acid sequence of SEQ ID NO: 20 is an amino acid other than cysteine;
(m) a DNA encoding a protein lacking a β-glucan synthesis activity, wherein the protein consists of an amino acid sequence in which one or several amino acids are deleted, substituted, or added in the amino acid sequence of SEQ ID NO: 20, wherein the amino acid corresponding to position 253 of the amino acid sequence of SEQ ID NO: 2 is tyrosine;
(n) a DNA consisting of the base sequence of SEQ ID NO: 19;
(o) a DNA consisting of a base sequence having 90% or more homology to the base sequence of SEQ ID NO: 4 and producing a protein having a β-glucan synthesis activity when transcribed and translated;
(p) a DNA consisting of the base sequence of SEQ ID NO: 4;
(q) a DNA consisting of a base sequence having 90% or more homology to the base sequence of SEQ ID NO: 15 and producing a protein having a β-glucan synthesis activity when transcribed and translated;
(r) a DNA consisting of the base sequence of SEQ ID NO: 15;
(s) a DNA consisting of a base sequence having 90% or more homology to the base sequence of SEQ ID NO: 6 and encoding a protein having a β-glucan synthesis activity; and
(t) a DNA consisting of the base sequence of SEQ ID NO: 6.

18. An isolated Genomic DNA of a β-glucan-deficient gene selected from the group consisting of the DNAs of (a) to (h) according to claim 17.

19. An isolated cDNA of a β-glucan-deficient gene selected from the group consisting of the DNAs of (i) to (n) according to claim 17.

20. An isolated Genomic DNA of a β-glucan synthesis gene selected from the group consisting of the DNAs of (o) to (r) according to claim 17.

21. An isolated cDNA of a β-glucan synthesis gene consisting of the DNA of (s) or (t) according to claim 17.

22. A method for breeding a barley having a genomic DNA of a β-glucan-deficient gene.

23. A method for breeding a barley having a genomic DNA of a β-glucan-deficient gene according to claim 22, comprising:
a selection step of selecting the barley by determining the barley as having a genomic DNA of a β-glucan-deficient gene when the base corresponding to position 4,275 of the genomic DNA consisting of the base sequence of SEQ ID NO: 4 located near the centromere of chromosome 7H of the barley is mutated from G to A; or
a selection step of selecting the barley by determining the barley as having a genomic DNA of a β-glucan-deficient gene when the base corresponding to position 2,385 of the genomic DNA consisting of the base sequence of SEQ ID NO: 4 located near the centromere of chromosome 7H of the barley is mutated from G to A.

24. A method for breeding a barley having a genomic DNA of a β-glucan-deficient gene according to claim 22, comprising:
an amplification step of amplifying a DNA fragment containing a base corresponding to position 4,275 of the base sequence of SEQ ID NO: 4, using a genomic DNA extracted from a barley as a template;
a detection step of cleaving the DNA fragment amplified in the amplification step with a restriction enzyme selected from the group consisting of TaqI, BanI, and NlaIV and detecting the cleaved DNA fragment; and
a selection step of selecting the barley by determining the barley as having a genomic DNA of a β-glucan-deficient gene when the DNA fragment is cleaved with the restriction enzyme TaqI or not cleaved with the restriction enzyme BanI or NlaIV in the detection step.

25. A method for breeding a barley having a genomic DNA of a β-glucan-deficient gene according to claim 22, comprising:
an amplification step of amplifying a DNA fragment containing a base corresponding to position 2,385 of the base sequence of SEQ ID NO: 4, using a genomic DNA extracted from a barley as a template;
a detection step of cleaving the DNA fragment amplified in the amplification step with a restriction enzyme Fnu4HI and detecting the cleaved DNA fragment; and
a selection step of selecting the barley by determining the barley as having a genomic DNA of a β-glucan-deficient gene when the DNA fragment is not cleaved between the bases corresponding to positions 2,386 and 2,387 of SEQ ID NO: 4 in the detection step.

26. A method for decreasing or abolishing β-glucan in barley kernels, comprising a step of suppressing the expression of the genomic DNA of a β-glucan synthesis gene according to claim 20.

27. A barley decreasing or deficient in β-glucan in kernels obtainable by the method according to claim 26.

28. A barley having the genomic DNA of a β-glucan-deficient gene according to claim 18 and bred by the breeding method according to claim 23.

29. A barley having the genomic DNA of a β-glucan-deficient gene according to claim 18 in a homozygous form and bred by the breeding method according to claim 23.

30. A transformant comprising a vector containing the genomic DNA of a β-glucan synthesis gene according to claim 20 so as to be expressible.

31. A transformant comprising a vector containing the cDNA of a β-glucan synthesis gene according to claim 21 so as to be expressible.

32. A method for producing an alcohol, comprising a step of fermenting kernels derived from the barley according to claim 27.

33. A method for producing a fermented food, comprising a step of fermenting kernels derived from the barley according to claim 27.

34. An animal feed composition comprising kernels derived from the barley according to claim 27.


SEQUENCE LISTING (28 sequences)

SEQ ID NO: 1 - DNA, 5140 bp, Hordeum vulgare
  misc_feature (1)..(5140): OUM125 bgl gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactctagtatttatttttactctctatctgcacaacaagcgctactacaattaacccaacaagcaccacgccaggttgacagtcaggataatttgatcttgaccggagtaagtactagtactaggtcggtgttaatcagagtaattattgcactagttaattaaaatttgagtaatccgagacaggtgcacgttagggccgggccaatgatggctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatgtcccttttttggcaccgaagaaatcctatgtacggcgagttttttctgcatctagttatgggtagatgtacgttagtttttgttgagcgttttatgtgctacatatatggagaaaaagagaaaaatattatcatgtcatgtcatgccatgccatgagaaggaggagaagaaaaagaagtgttgccaccgctcgaatgccttttttttctttcggaaggatgcgtgagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtataacgagaaaagaggcccattaatagagcaacgaatcgagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctatactgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagtcgagagtgcaaccaccacccagagccagccaagcacatgcatgcttctcttctcgtctcgtcgtgtggccagcagcgcattcatgctattgctgtgacgagggaggaatggtagttggggtggtcctttccccccgacagcactacagcctccactttatgacccatttaattcaccggccctgctttgttgtaaccgccttctcatctcaatcaatcattcattcattcataagtttactcactctttgttactactcgaaccactaatcaggaaggagtaggagtaatgcagatttactattgacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcgacaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 2 - protein, 947 aa, Hordeum vulgare
  misc_feature (1)..(947): OUM125 bgl
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYAAAAEGIVADEATIVEAVNVTAAAFEKKTGWGKEIGWVYDTVTEDVVTGYRMHIKGWRSRYCSIYPHAFIDTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 3 - DNA, 2844 bp, Hordeum vulgare
  misc_feature (1)..(2844): OUM125 bgl gene cDNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcgacaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 4 - DNA, 5140 bp, Hordeum vulgare
  misc_feature (1)..(5140): Akashinriki HvCslF6 gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactctagtatttatttttactctctatctgcacaacaagcgctactacaattaacccaacaagcaccacgccaggttgacagtcaggataatttgatcttgaccggagtaagtactagtactaggtcggtgttaatcagagtaattattgcactagttaattaaaatttgagtaatccgagacaggtgcacgttagggccgggccaatgatggctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatgtcccttttttggcaccgaagaaatcctatgtacggcgagttttttctgcatctagttatgggtagatgtacgttagtttttgttgagcgttttatgtgctacatatatggagaaaaagagaaaaatattatcatgtcatgtcatgccatgccatgagaaggaggagaagaaaaagaagtgttgccaccgctcgaatgccttttttttctttcggaaggatgcgtgagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtataacgagaaaagaggcccattaatagagcaacgaatcgagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctatactgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagtcgagagtgcaaccaccacccagagccagccaagcacatgcatgcttctcttctcgtctcgtcgtgtggccagcagcgcattcatgctattgctgtgacgagggaggaatggtagttggggtggtcctttccccccgacagcactacagcctccactttatgacccatttaattcaccggccctgctttgttgtaaccgccttctcatctcaatcaatcattcattcattcataagtttactcactctttgttactactcgaaccactaatcaggaaggagtaggagtaatgcagatttactattgacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 5 - protein, 947 aa, Hordeum vulgare
  misc_feature (1)..(947): Akashinriki HvCslF6
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYAAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 6 - DNA, 2844 bp, Hordeum vulgare
  misc_feature (1)..(2844): Akashinriki HvCslF6 gene cDNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 7 - DNA, 5140 bp, Hordeum vulgare
  misc_feature (1)..(5140): Nishinohoshi HvCslF6 gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgttctgctcccacaactaaacagaaactccctatatctgcgtcacactcaacaattaatccaactaagtctctctactactctagtatttatttttactctctatctgcacaacaagcgctactacaattaacccaacaagcaccacgccaggttgacagtcaggataatttgatcttgaccggagtaagtactagtactaggtcggtgttaatcagagtaattattgcactagttaattaaaatttgagtaatccgagacaggtgcacgttagggccgggccaatgatggctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatgtcccttttttggcaccgaagaaatcctatgtacggcgagttttttctgcatctagttatgggtagatgtacgttagtttttgttgagcgttttatgtgctacatatatggagaaaaagagaaaaatattatcatgtcatgtcatgccatgccatgagaaggaggagaagaaaaagaagtgttgccaccgctcgaatgccttttttttctttcggaaggatgcgtgagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtataacgagaaaagaggcccattaatagagcaacgaatcgagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctatactgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagtcgagagtgcaaccaccacccagagccagccaagcacatgcatgcttctcttctcgtctcgtcgtgtggccagcagcgcattcatgctattgctgtgacgagggaggaatggtagttggggtggtcctttccccccgacagcactacagcctccactttatgacccatttaattcaccggccctgctttgttgtaaccgccttctcacctcaatcaatcattcattcattcataagtttactcactctttgttactactcgaaccactaatcaggaaggagtaggagtaatgcagatttactattaacagttaaaggagtaaaaagaaggaagcacaattacagaaccttgtttttttttactactgtacgtaaggtgtaagaatggagcgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 8 - protein, 947 aa, Hordeum vulgare; identical to SEQ ID NO: 5
  misc_feature (1)..(947): Nishinohoshi HvCslF6

SEQ ID NO: 9 - DNA, 2844 bp, Hordeum vulgare
  misc_feature (1)..(2844): EU267181 HvCslF6 mRNA to cDNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctagatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 10 - protein, 947 aa, Hordeum vulgare; identical to SEQ ID NO: 5
  misc_feature (1)..(947): EU267181 HvCslF6

SEQ ID NO: 11 - DNA, 22 bp, Artificial Sequence
  Forward primer
gccaagaccaagtacgagaagc

SEQ ID NO: 12 - DNA, 23 bp, Artificial Sequence
  Reverse primer
tgttcttggagaagaagatctcg

SEQ ID NO: 13 - DNA, 2279 bp, Hordeum vulgare
  misc_feature (1)..(2279): TR251 HvCslF6 promoter
atcttgtttttgcgggacagcttacgaattactttttattttgcggtacagcttacgaattacttaacttgatggagcagttcagcaaagaaattgagcaaaaatcatttgcagagtcaaggggtgagcttgtgggcgtcaatcaggtgattcgtctcctgtccacacatagacgcgcgcgcacacataagcaagtgagtagtaggtgattacattaccgtcggcgtgaagcgttaaaattgcgtccgttctctcccgcctcctcgtaaatgctttgggactcgttgattgtagcagtggtagtttatcatgaatgctggggctagcgtgcccgcatatgtagttggatcgtcgatgcaacagcaaggccggttaattactcctccggcctctccataccagtgcacggctcgccacgatcgttttcttgtggtatgttgtccttttgtgtgttgtgtggcggtgtggttatagttggaggtcgtgacagaacccattagttgtccggcgcgcgcgcgtcattgtcctaacaactggcctaatttgcaaattgctctagacctcatgcttgattcctcccgttagttcagggagctccatcagcacggattacatgctcctccccgtccccgccgtgaatctctcatcggatacgcgcgcgacgttccccgccgtgactctctcaatctctcatcggatacgcgcgggacgttcctatagggacgtcgtaacgggcagaagggcaagcctccttccggccaggggaccaatgcattccttctcgtgaagcatttgctcgccaaagccaaagccaaagccaaagccacaaggggaaaaagagagagaaggaggcggtagaaccggaaagggctctgcatgtctacctttcgctgtgaccaaaattactcttcccttccgtggccctgctgcatgagcatggccctccgcttttcaaccagcaagacaagaatatgtcgcaattgctcctactagtgttaaaccgatggatcaatcctccaaaatgcatcccccaaccagacccaacttgcgcataaacatcaatgtgctaaaaattccatggacagaagagagcgccgcatcgcatttgccggtgggctgcaaggccacagcggagcaaagaggcttgtacggtactcaaaccagctcactgatgcatggcgatgctacagggactcacgggccacgctggctgggagagagcagcagaaaaggcgcaagaaaagaatctttctctcaatcaagggcaacaaccaagcacaagctcacaaaccccccaccaagttaagaaaaagaaaaaaaaatacaggcacttcgcataacaaacaaactaatcatcatcaccactcatcgcccccggcacctttattctacgcccagctcctctctcattacacttctaacatatataacaatcaatcggtggaggaggagggaccaattaattaatggattagtaataacaattcttatgcgagcaaaaatatacgtactcctgtactaataagaatcggaaatgcaagctaatcagagctactgcgtacgtactagttttcttgcgcggggagggcatcacaaatcacatgggacacgcaggggcaggaatatgagtggaagcaacctggtaggtaggtgaggtgcggtgcggtgcgggcatatgataagcatatgggctgggccgagcatacacgcgtacatgcattgcatttgcatacaccgaaggaagtgcttggcaccacctacatataaacgcacccgcacaccgctcaaacacaatttacaacccgcacacagctccaacccgttggttggatagctcagcaagacaagcgagctgagcagagctttgctttgcttgaaccccctaccgacacttcttgtgcatcccagcccataaatcccccacgtactttactcgtcatttctcgcacctcctcctccccctccccctcgcctacattcattcattgcttccttttctctctccctgcacatgctaaagcctgcctcggccattgcctgagcctgccattgttggaccttggacctgttctccttgtcgtaaaggcaaaggagagcgcgtgcattgaggacgacggccatggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctgg

SEQ ID NO: 14 - DNA, 2271 bp, Hordeum vulgare
  misc_feature (1)..(2271): CDC-Bold HvCslF6 promoter
atcttgtttatgcgggacagcttacgaattactttttattttgcagtacagcttacgaattacttaacttgatggagcagttcagcaaagaaattgagcaaaaatcatttgcagagtcaaggggtgagcttgtgggcgtcaatcaggtgattcgtctcctgtccacacatagacgcgcgcgcacacataagcaagtgagtagtaggtgattacattaccgtcggcgtgaagcgttaaaattgcgtccgttctctcccgcctcctcgtaaatgctttgggactcgttgattgtagcagtggtagtttatcatgaatgctggggctagcgtgcccgcatatgtagttggatcgtcgatgcaacagcaaggccggttaattactcctccggcctctccataccagtgcacggctcgccacgatcgttttcttgtggtatgttgtccttttgtgtgttgtgtggcggtgtggttatagttggaggtcgtgacagaacccattagttgtccggcgcgcgcgcgtcattgtcctaacaactggcctaatttgcaaattgctctagacctcatgcttgattcctcccgttagttcagggagctccatcagcacggattacgtgctcctccccgtccccgccgtgaatctctcatcggatacgcgcgcgacgttccccgccgtgactctctcatcggatacgcgcgggacgttcctatagggacgtcgtaacgggcagaagggcaagcctccttccggccaggggaccaatgcattccttctcgtgaagcatttgctcgccaaagccaaagccaaagccaaagccacaaggggaaaaagagagagaaggaggcggtagaaccggaaagggctctgcatgtctacctttcgctgtgaccaaaattactcttcccttccgtggccctgctgcatgagcatggccctccgcttttcaaccagcaagacaagaatatgtcgcaattgctcctactagtgttaaaccgatggatcaatcctccaaaatgcatcccccaaccagacccaacttgcgcataaacatcaatgtgctaaaaattccatggacagaagagagcgccgcatcgcatttgccggtgggctgcaaggccacagcggagcaaagaggcttgtacggtactcaaaccagctcactgatgcatggcgatgctacagggactcacgggccacgctggctgggagagagcagcagaaaaggcgcaagaaaagaatctttctctcaatcaagggcaacaaccaagcacaagctcacaaaccccccaccaagttaagaaaaagaaaaaaaaatacaggcacttcgcataacaaacaaactaatcatcatcaccactcatcgcccccggcacctttattctacgcccagctcctctctcattacacttctaacatatataacaatcaatcggtggaggaggagggaccaattaattaatggattagtaataacaattcttatgcgagcaaaaatatacgtactcctgtactaataagaatcggaaatgcaagctaatcagagctactgcgtacgtactagttttcttgcgcggggagggcatcacaaatcacatgggacacgcaggggcaggaatatgagtggaagcaacctggtaggtaggtgaggtgcggtgcggtgcgggcatatgataagcatatgggctgggccgagcatacacgcgtacatgcattgcatttgcatacaccgaaggaagtgcttggcaccacctacatataaacgcacccgcacaccgctcaaacacaatttacaacccgcacacagctccaacccgttggttggatagctcagcaagacaagcgagctgagcagagctttgctttgcttgaaccccctaccgacacttcttgtgcatcccagcccataaatcccccacgtactttactcgtcatttctcgcacctcctcctccccctccccctcgcctacattcattcattgcttccttttctctctccctgcccatgctaaagcctgcctcggccattgcctgagcctgccattgttggaccttggacctgttctccttgtcgtaaaggcaaaggagagcgcgtgcattgaggacgacggccatggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctgg

SEQ ID NO: 15 - DNA, 5140 bp, Hordeum vulgare
  misc_feature (1)..(5140): Sachiho Golden HvCslF6 gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactctagtatttatttttactctctatctgcacaacaagcgctactacaattaacccaacaagcaccacgccaggttgacagtcaggataatttgatcttgaccggagtaagtactagtactaggtcggtgttaatcagagtaattattacactagttaattaaaatttgagtaatccgagacaggtgcacgttagggccgggccaatgatggctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatgtcccttttttggcaccgaagaaatcctatgtacggcgagttttttctgcatctagttatgggtagatgtacgttagcttttgttgagcgttttatgtgctacatatatggagaaaaagagaaaaatattatcatgtcatgtcatgccatgccatgagaaggaggagaagaaaaataagtgttgccaccgctcgaatgccttttttttctttcggaaggatgcgtaagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtataacgagaaaagaggcccattaatagagcaacgaatccagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctatactgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagtcgagagtgcaaccaccacccagagccagccaagcacatgcatgcttctcttctcgtctcgtcgtgtggccagcagcgcattcatgctattgctgtgacgagggaggaatggtggttggggtggtcctttccccccgacagcactacagcctccactttatgacccatttaattcaccggccctgctttgttgtaaccgccttctcacctcaatcaatcattcattcattcataagtttactcactctttgttactactcgaaccactaatcaggaaggagtaggagtaatgcagatttactattaacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 16 - DNA, 2844 bp, Hordeum vulgare; identical to SEQ ID NO: 6
  misc_feature (1)..(2844): Sachiho Golden HvCslF6 gene cDNA

SEQ ID NO: 17 - protein, 947 aa, Hordeum vulgare; identical to SEQ ID NO: 5
  misc_feature (1)..(947): Sachiho Golden HvCslF6

SEQ ID NO: 18 - DNA, 5140 bp, Hordeum vulgare
  misc_feature (1)..(5140): KM27 bgl gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactctagtatttatttttactctctatctgcacaacaagcgctactacaattaacccaacaagcaccacgccaggttgacagtcaggataatttgatcttgaccggagtaagtactagtactaggtcggtgttaatcagagtaattattacactagttaattaaaatttgagtaatccgagacaggtgcacgttagggccgggccaatgatggctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatgtcccttttttggcaccgaagaaatcctatgtacggcgagttttttctgcatctagttatgggtagatgtacgttagcttttgttgagcgttttatgtgctacatatatggagaaaaagagaaaaatattatcatgtcatgtcatgccatgccatgagaaggaggagaagaaaaataagtgttgccaccgctcgaatgccttttttttctttcggaaggatgcgtaagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtataacgagaaaagaggcccattaatagagcaacgaatccagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctaccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctatactgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagtcgagagtgcaaccaccacccagagccagccaagcacatgcatgcttctcttctcgtctcgtcgtgtggccagcagcgcattcatgctattgctgtgacgagggaggaatggtggttggggtggtcctttccccccgacagcactacagcctccactttatgacccatttaattcaccggccctgctttgttgtaaccgccttctcacctcaatcaatcattcattcattcataagtttactcactctttgttactactcgaaccactaatcaggaaggagtaggagtaatgcagatttactattaacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 19 - DNA, 2844 bp, Hordeum vulgare
  misc_feature (1)..(2844): KM27 bgl gene cDNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtggccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctaccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacgccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgattacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 20 - protein, 947 aa, Hordeum vulgare
  misc_feature (1)..(947): KM27 bgl gene
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFYRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYAAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 21 - DNA, 20 bp, Artificial Sequence
  Synthetic construct (HvCSLF6_MPP2-1F)
atcaaggagcccatcctctc

SEQ ID NO: 22 - DNA, 20 bp, Artificial Sequence
  Synthetic construct (HvCSLF6_MPP2-1R)
ttgatcctggccttgaactc

SEQ ID NO: 23 - DNA, 5124 bp, Hordeum vulgare
  misc_feature (1)..(5124): TR251 HvCslF6 gene genomic DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtagccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactatagtatttatttttactctctatctgcacaacaagcgctactacaattaacccaacaagcaccacgccaggttgacagtcaggataatttgatcttgaccggagtaagtactagtactaggtcggtgttaatcagagtaattattgcactagttaattaaaatttgagtaatccgagacaggtgcacgttagggccgggccaatgatcgctcgaatccacccaaaatagcgcgtcccggtgtgggctgtcggctcggtgcttcttccttccattttactagtcgcagtcactgcagcttgggcccacgggaggggacgttagccgttgggcctgcctggcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatggcccttttttggcaccgaagaaatcctatgtacggcgagttttttctgcatctagttatgggtagatgtacgttagtttttgttgagcgttttatgtgctacatatatggagaaaaagagaaaaatattatcatgtcatgtcatgccatgagaagaaaaagaagtgttgccaccgctcgaatgcctcttttttctttcggaaggatgcgtgagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtaacgagaaaagaggcccattaatagagcaacgaatccagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctataccgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagttgagagtgcaaccaccacccagagccagccaagcacatgcatgcttctcttctcgtctcgtcgtgtggccagcagcgcattcatgctattgctgtgacgagggaggaatggtggttggggtggtcctttccccccgacagcactacagcctccactttatgacccatttaattcatcggccctgctttgttgtaaccgccttctcacctcaatcaatcattcattcattcataagtttactcactctttgttactactcgaaccactaatcaggaaggagtaggagtaatgcagatttactattaacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacaccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgatcacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 24 - DNA, 2844 bp, Hordeum vulgare
  misc_feature (1)..(2844): TR251 HvCslF6 gene cDNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtagccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccgggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacaccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgatcacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgcatggtcaccatggcaagaagttggtcgacacagggctctatggctggctccattga

SEQ ID NO: 25 - protein, 947 aa, Hordeum vulgare
  misc_feature (1)..(947): TR251 HvCslF6
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYTAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 26 - DNA, 5140 bp, Hordeum vulgare; identical to SEQ ID NO: 4
  misc_feature (1)..(5140): CDC-Bold HvCslF6 gene genomic DNA

SEQ ID NO: 27 - DNA, 2844 bp, Hordeum vulgare; identical to SEQ ID NO: 6
  misc_feature (1)..(2844): CDC-Bold HvCslF6 gene cDNA

SEQ ID NO: 28 - protein, 947 aa, Hordeum vulgare; identical to SEQ ID NO: 5
  misc_feature (1)..(947): CDC-Bold HvCslF6